(19) United States
(12) Patent Application Publication
(10) Pub. No.: US 2005/0119458 A1
(43) Pub. Date: Jun. 2, 2005

(54) Novel human proton-gated channels

(75) Inventors: Phillippe Sequela, Outermont, Quebec (CA); Kazimierz Babinski, Dorval, Quebec (CA); Naima Abbadi, Montreal (CA); Stefano Catarsi, Montreal (CA)

Correspondence Address: Jean C Baker;Quarles & Brady, 411 E Wisconsin Avenue, Suite 2040, Milwaukee, WI, 53202-1197, US

(73) Assignee: McGill University, Montreal PQ (CA)

(21) Appl. No.: 10/484,187

(22) PCT Filed: Jul. 18, 2002
(86) PCT No.: PCT/CA02/01120

(30) Foreign Application Priority Data
Jul. 18, 2001 (CA) 2352702

(51) Int. Cl. C12Q 1/68; G01N 33/53; G01N 33/567; C07K 14/705; C07K 16/28

(52) U.S. Cl. 530/350; 435/69.1; 435/320.1; 435/325; 536/23.5; 514/12; 514/44; 435/7.2; 435/6; 530/388.22

(57) ABSTRACT

The present invention provides a novel human proton-gated ion channel (hASIC1B) and polynucleotides which identify and encode hASIC1B. The invention also provides genetically engineered expression vectors and host cells comprising the nucleic acid sequences encoding hASIC1B and a method for producing hASIC1B. The invention also provides for use of hASIC1B, and agonists, antibodies or antagonists specifically binding hASIC1B, in the prevention and treatment of diseases associated with expression of hASIC1B. Additionally, the invention provides for the use of antisense molecules to polynucleotides encoding of hASIC1B for the treatment of diseases associated with the expression of hASIC1B. The invention also provides diagnostic assays, which utilize the polynucleotides, or fragments or the complements thereof, and antibodies specifically binding hASIC1B.

### FIELD OF INVENTION

In mammals, the pH of the extracellular compartment, including interstitial fluids and blood, is strictly regulated and maintained at a constant value of 7.4. Acid sensing is a specific kind of chemoreception that plays a critical role in the detection of nociceptive pH imbalances occurring, for example, in conditions of cramps, trauma, inflammation or hypoxia (Lindahl, Adv Neurol 1974; 4: 45)). In mammals, a population of small-diameter primary sensory neurons in the dorsal root ganglia and trigeminal ganglia express specialized pH-sensitive surface receptors activated by increase of extracellular proton concentration (Bevan and Yeats, J Physiol (Lond) 1991; 433: 145). Acid sensitivity of sensory as well as central neurons is mediated by a family of proton-gated cation channels structurally related to C. elegans degenerins (DEG) and mammalian epithelial sodium channels (ENaC). This invention relates to these Acid Sensing Ion Channels (ASIC) and specifically reports the discovery of a novel member of this class of receptor-channels, its association with other channel subunits and uses thereof.

### BACKGROUND OF THE INVENTION

Tissue acidosis is associated with a number of painful physiological (e.g. cramps) and pathological conditions (e.g. inflammation, intermittent claudication, myocardial infarction). Experimentally, similar painful events can be reproduced by infusing low pH solutions into skin or muscle. Furthermore, the prolonged intradermal infusion of low pH solutions can mimic the characteristic hyperalgesia of chronic pain. To further charaterize the effects of protons and their relation to pain, low pH solutions were applied to patch-clamped central and peripheral sensory neurons. Inward currents were induced when pH was dropped to acidic values, providing evidence for the existence of proton-activated ion channels. Several types of native currents were observed in sensory neurons from rat and human trigeminal and dorsal root ganglia: rapidly inactivating currents; non-inactivating currents; and biphasic currents displaying a rapidly inactivating current followed by non-inactivating current.

Other differences regarding ion selectivities were also reported. These results suggested the existence of several proton-gated ion channels. The prolonged pain induced by tissue acidification is most likely associated with a non-inactivating proton-gated ion channel.

Cloned Proton-Gated Ion Channels

The mammalian proton-gated cation channels have recently been cloned and named <<ASIC>> for Acid Sensing Ion Channels. Sequence analysis identifies them as members of the DEG/ENaC superfamily of ion channels. The putative membrane topology of ASIC receptors predicts two transmembrane spanning domains with both N— and C— termini in the intracellular compartment, as shown for the epithelial sodium channels. Four sub-classes of ASIC receptors have been identified:
1. ASIC1 ion channels display rapidly inactivating inward currents (Waldmann et al., Nature 1997; 386: 173)
2. ASIC2 ion channels display slowly inactivating inward currents (Brassilana et al., J Biol Chem 1997; 272: 28819).
3. ASIC3 ion channels display biphasic inward currents with an initial rapidly inactivating component, followed by a sustained non-inactivating current (Waldmann et al., J Biol Chem 1997; 272: 20975; Babinski et al., J Neurochem 1999; 72: 51)
4. ASIC4 ion channels contain characteristic ASIC-like sequence motifs but do not appear to be activated by protons in homomultimeric association.
   Families of ASIC Receptors Created by Alternative Splicing of mRNAs

A common feature of these ion channels is the existence of alternative splice variants, which display important functional differences. Indeed, the replacement of the first 185 amino acids of ASIC1 (hereinafter named ASIC1A) by a distinct new sequence of 172 amino acids generates a new channel, ASIC1B, which has similar current kinetics as ASIC1A but needs lower pH values for activation (pH₅₀ of 6.2 and 4.5, respectively for ASIC1A and ASIC1B). Also, it appears that ASIC1B is specifically expressed in rat dorsal root ganglia. A similar situation is also observed with rat ASIC2 (hereinafter named ASIC2A), where the replacement of the first 185 amino acids by a distinct new sequence of 236 amino acids generates another ASIC ion channel subunit, ASIC2B. When expressed alone as a homomultimer in mammalian cells or Xenopus oocytes, ASIC2B does not appear to be activated by low pH solutions. However, coexpression of ASIC2B with other ASIC subunits (e.g. ASIC2A, ASIC3) gives rise to heteropolymeric ion channels with distinct properties such as novel ion selectivities or pH₅₀ values (Lingueglia et al., J Biol Chem 1997: 272: 30 29778). ASIC3, which has been identified in human, also appears to exist in various forms. Indeed, DRASIC is an ASIC3-like channel identified in rat, which displays 85% identity with the human ASIC3 sequence and has similar biphasic current kinetics. However, important differences regarding tissue distribution, ion selectivities and pH₅₀ suggest that DRASIC might not be the human orthologue of ASIC3 but rather a different subtype. Furthermore, the existence of two 3′ splice variants of human ASIC3 (ASIC3B and 3C, recently submitted to GenBank) have been reported but differences in function have yet to be documented. Alternative splicing, therefore, appears like an important mechanism for increasing the diversity of ASIC receptors, which most probably assume critical roles in the nervous system, such as neurotransmission, nociception or mechanosensation (see below). Because of the great, differences between the existing splice variants, the actual functional characteristics of the new splice variants is unpredictable and might prove to be completely different from any known ASIC receptor.

### SUMMARY OF THE INVENTION

The present invention reports the discovery of the human ASIC1B receptor (hereinafter referred to as hASIC1B), which shows distinct features from the previously published rat ASIC1B. Also contemplated within the scope of this invention is the potential involvement of this new subunit in neurotransmission and/or nociception and/or mechanosensation and/or any other neurological and/or metabolic processes in normal and pathophysiological conditions. This invention seeks also to cover any uses of this new subunit as a therapeutic target, including but not limiting to drug screening technologies (i.e. screening for channel antagonists, agonists and/or modulators), diagnostic marker, gene therapies. Also within the scope of the present invention is the heteropolymerization of the hASIC1B subunits with each other and/or with one or more subunits of the ASIC family from any species, including but not limiting to ASIC1, ASIC1A, BNaC2, ASIC1B, ASIC2A, ASIC2B, MDEG, MDEG1, MDEG2, BNC1, BNaC1, DRASIC, ASIC3, ASIC4, SPASIC or any variants thereof, as well as heteropolymerization of hASIC1B with any other members of the Degenerin and EnaC family from any species.

The object of this invention is to provide the preferred primary sequence of the polynucleotide molecule (SEQ ID No.1) encoding the full length hASIC1B polypeptide molecule (SEQ ID No.2). Still another object of this invention is to provide a partial genomic polynucleotide sequence of hASIC1B deduced from the non-characterized sequences deposited in GenBank under Accession NO: AC025154, AC074032, and AC025361. In particular, the sequence of the characteristic and distinctive first exon of hASIC1B contained within one uninterrupted contig in clone AC025154.

The invention additionally features nucleic acid sequences encoding polypeptides, oligonucleotides, peptide nucleic acids (PNA), fragments, portions or antisense molecules thereof, and expression vectors and host cells comprising polynucleotides that encode hASIC1B. The present invention also features antibodies which bind specifically to hASIC1B, and pharmaceutical compositions comprising substantially purified hASIC1B. The invention also features use of agonists and antagonists of hASIC1B.

### DESCRIPTION OF THE FIGURES

The following drawings, figures and tables are illustrative of the embodiments of the invention and are not meant to limit the scope of the invention as encompassed by the claims.

FIG. 1: depicts the nucleotide sequence SEQ ID NO: 1 and the deduced amino acid sequence SEQ ID NO: 2 of the full-length hASIC1B. Arrows indicate the intron/exon splice sites.

FIG. 2: illustrates the structural comparison of the amino acid sequence of hASIC1B (SEQ ID NO: 2) with amino acid sequences of cloned ASIC family members, including human ASIC1A, human ASIC2A, human ASIC2B, human ASIC3, human ASIC4 and rat ASIC1B. Conserved amino acids are boxed in grey and dashes represent gaps inserted for best alignment score.

FIG. 3: illustrates the specific structural comparison of the amino acid sequence of hASIC1B (SEQ ID NO: 2) with amino acid sequence of rat ASIC1B. Conserved amino acids are boxed in grey and dashes represent gaps inserted for best alignment score.

FIG. 4: illustrates the specific structural comparison of the amino acid sequence of hASIC1B (SEQ ID NO: 2) with amino acid sequence of human ASIC4. Conserved amino acids are boxed in grey and dashes represent gaps inserted for best alignment score.

FIG. 5: illustrates pH-activated inward currents recorded using voltage clamped COS cells expressing either hASIC1B, rat ASIC1B or human ASIC1A.

FIG. 6: illustrates pH-activated inward currents recorded using voltage clamped Xenopus oocytes expressing hASIC1B.

FIG. 7: illustrates the proton dose response curves of hASIC1B and human ASIC1A, expressed in COS cells.

FIG. 8: illustrates the I/V curves of hASIC1B and human ASIC1A, expressed in COS cells.

### DETAILED DESCRIPTION OF THE INVENTION

Before the present nucleotide sequences, proteins and methods are described, it is understood that this invention is not limited to the particular methodology, protocols, cell lines, vectors, and reagents described, as these may vary. It is also to be understood that the terminology used herein is for the purpose of describing particular emobodiments only, and is not intended to limit the scope of the present invention which will be limited only by the appended claims. It must be noted that as used herein and in the appended claims, the singular forms “a”, “an”, and “the” include plural reference unless the context clearly dictates otherwise. Thus, for example, reference to “a host cell” includes a plurality of such host cells; reference to “the antibody” is a reference to one or more antibodies and equivalents thereof known to those skilled in the art, and so forth.

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods, devices, and materials are now described. All publications mentioned herein are incorporated by reference for the purpose of describing and disclosing the cell lines, vectors, and methodologies which are reported in the publications which might be used in connection with the invention.

Unless specified otherwise, the term “hASIC1B” used hereinafter and before encompasses all variants (as defined below) of hASIC1B.

Definitions

“Polynucleotide” as used herein refers to single- or double-stranded molecules which may be “deoxyribonucleic acid” (DNA), comprised of the nucleotide bases A, T, C and G, or “ribonucleic acid” (RNA), comprised of bases A, U (substitutes for T), C and G. Polynucleotides may represent a coding strand or its complement, the sense or anti-sense strands. Polynucleotides may be identical in sequence to the sequence which is naturally occurring or may include alternative codons which encode the same amino acid as that which is found in the naturally occurring sequence (Lewin: “Genes V”, Chapter 7; Oxford University Press, 1994). Furthermore, polynucleotides may include codons which represent conservative substitutions of amino acids. The term “polynucleotide” will also include all possible alternate forms of DNA or RNA, such as genomic DNA (both introns and exons), complementary DNA (cDNA), cRNA, messenger RNA (mRNA), and DNA or RNA prepared by partial or total chemical synthesis from nucleotide bases, including modified bases, such as tritylated bases and unusual bases such as inosine. Polynucleotides will also embrace all chemically, enzymatically or metabolically modified forms of DNA or RNA, as well as the chemical forms of DNA and RNA characteristic of viruses.

The term “oligonucleotide” or “oligo” will refer to short polynucleotides, typically between 10 to 40 bases in length.

“Polypeptide” refers to a molecule comprised of two or more amino acids joined to each other by peptide bonds or modified peptide bonds (i.e. isosteres). Amino acids include all 20 naturally gene-encoded amino acids as well as naturally or chemically modified amino acids. Polypeptides refer to both short chains of amino acids, commonly referred to as peptides, oligopeptides, or oligomers, and to longer chains, commonly referred to as proteins. Thus, “amino acid sequence” as used herein refers to an oligopeptide, peptide, polypeptide, or protein molecule and fragments or portions thereof, corresponding to a naturally occurring or synthetic molecule. Where “amino acid sequence” is recited herein to refer to an amino acid sequence of a naturally occurring protein molecule, “amino acid sequence” and like terms, such as “polypeptide” or “protein” are not meant to limit the amino acid sequence to the complete, native amino acid sequence associated with the recited protein molecule. Furthermore, polypeptides will also include amino acid sequences modified either by natural processes, such as posttranslational processing, or by chemical modification techniques, which are well known in the art. A given polypeptide may contain many types of modifications or a given modification may be present in the same or varying degrees at several sites in a given polypeptide. Modifications can occur anywhere in the polypeptide, including but not limited to, the peptide backbone, the amino acid side-chains and the amino or carboxyl termini. All the above referred to modifications as well as their practice are well described in the research literature, both in basic texts and detailed monographs (“Proteins: Structure and Molecular Properties”; Creighton T E, Freeman W H, ₂nd Ed., New-York,1993; “Posttranslational Covalent Modification of Proteins”, Johnson B C, ed., Academic Press, New-York, 1983; Also: Seiter et al., Meth Enzymol 1990; 182: 626, and Rattan et al., Ann NY Acad Sci 1992; 663: 48).

“Peptide nucleic acid”, as used herein, refers to a molecule which comprises an oligonucleotide to which an amino acid residue, such as lysine, and an amino group have been added. These small molecules, also designated anti-gene agents, stop transcript elongation by binding to their complementary strand of nucleic acid (Nielsen et al. Anticancer Drug Des 1993; 8: 53). hASIC1B, as used herein, refers to the amino acid sequences of substantially purified hASIC1B obtained from human, whether natural, synthetic, semi-synthetic, or recombinant.

The term “variant” as used herein is a polynucleotide or polypeptide that differs from a reference polynucleotide or polypeptide, respectively. A typical variant of a polynucleotide differs in nucleotide sequence from another reference polynucleotide. Differences in the nucleotide sequence of the variant may or may not alter the amino acid sequence of a polypeptide encoded by the reference polynucleotide. Nucleotide changes may result in amino acid substitutions, additions, insertions, deletions, fusions, and truncations in the polypeptide encoded by the reference sequence, as discussed below. A typical variant of a polypeptide differs in amino acid sequence from another reference polypeptide. Generally, differences are such that the sequences of the reference polypeptide and the variant are closely similar overall and, in many regions, identical. A variant and reference polypeptide may differ in amino acid sequence by one or more substitutions, additions, insertion, deletions in any combination. A substituted or inserted amino acid residue may or may not be one encoded by the genetic code. A variant of a polynucleotide or polypeptide may be naturally occurring such as allelic or pseudoallelic variant, including polymorphisms or mutations at one or more bases, or it may be a variant that is not known to occur naturally. Non-naturally occurring variants of polynucleotides and polypeptides may be made by mutagenesis techniques or by direct synthesis. The term “mutant” is encompassed by the above definition of non-natural variants.

“splice variants” as referred to hereinafter are variants, which result from the differential or alternative splicing and assembly of exons present in a given gene. Typically, the encoded proteins will display total identity in most regions, but lower identity in the specific region encoded by different exons.

A “deletion”, as used herein, refers to a change in either amino acid or nucleotide sequence in which one or more amino acids or nucleotide residues, respectively, are absent, as compared to a reference polypeptide or polynucleotide.

An “insertion” or “addition”, as used herein, refers to a change in an amino acid or nucleotide sequence resulting in the addition of one or more amino acid or nucleotide residues, respectively, as compared to a reference polypeptide or polynucleotide.

A “substitution”, as used herein, refers to the replacement of one or more amino acids or nucleotides by different amino acids or nucleotides, respectively, as compared to a reference polypeptide or polynucleotide.

The term “derivative”, as used herein, refers to the chemical modification of a nucleic acid encoding hASIC1B or the encoded hASIC1B. Illustrative of such modifications would be replacement of hydrogen by an alkyl, acyl, or amino group. A nucleic acid derivative would encode a polypeptide which may or may not retain some or all of the essential biological characteristics of the natural molecule.

The term “identity” as used herein refers to a measure of the extent of identical nucleotides or amino acids that two or more polynucleotide or amino acid sequences have in common. In general, the sequences are aligned so that the highest order match is obtained, referred to as the “alignment”. Such optimal alignments make use of gaps, which are inserted to maximize the number of matches using local homology algorithms, such as the Smith-Waterman alignment. The terms “identity”, or “similarity”, or “homology”, or “alignment” are well known to skilled artisans and methods to perform alignments and measure identity are widely described and taught in the literature: Dayhoff et al., Meth Enzymol 1983; 91: 524—Lipman D J and Pearson W R, Science 1985; 227: 1435—Altschul et al., J Mol Biol 1990; 215: 403.—Pearson W R, Genomics 1991; 11: 635.—Gribskov M and Devreux J, eds. (1992) Sequence Analysis Primer, WH Freeman & Cie, New-York.—Altschul et al., Nature Gen 1994; 6: 119. Furthermore, methods to perform alignments and to determine identity and similarity are codified in computer programs and software packages, some of which may also be web-based and accessible on the internet. Preferred software includes but is not limited to BLAST (Basic Local Alignment Search Tools), including Blastn, Blastp, Blastx, tBlastn (Altschul et al., J Mol Biol 1990; 215: 403), FastA and TfastA (Pearson and Lipman, PNAS 1988; 85: 2444), Lasergene99 (DNASTAR, Madison Wis.), Omiga 2.0 or MacVector (Oxford Molecular Group, Cambridge, UK), Wisconsin Package (Genetic Computer Group (GCG), Madison, Wis.), Vector NTI Suite (InforMax Inc., N.Bethesta, Md.), GeneJockey II (Biosoft, Cambridge, UK).

As an illustration, by a polynucleotide having a nucleotide sequence with at least, for example, 95% “identity” to a reference nucleotide sequence of SEQ ID NO: 1, it is intended that the nucleotide sequence of the polynucleotide is identical to the reference sequence except that the polynucleotide sequence may include up to five point mutations, or divergent nucleotides, per 100 nucleotides of the reference nucleotide sequence of SEQ ID NO: 1. In other words, to obtain a polynucleotide having a nucleotide sequence at least 95% identical to a reference nucleotide sequence, up to 5% of the nucleotides in the reference sequence may be deleted or substituted with another nucleotide, or a number of nucleotides up to 5% of the total nucleotides in the reference sequence may be inserted into the reference sequence.

These mutations of the reference sequence may occur at the 5′ or 3′ terminal positions of the reference nucleotide sequence or anywhere between those terminal positions, interspersed either individually among nucleotides in the reference sequence or in one or more continuous groups within the reference sequence.

Similarly, by a polypeptide having an amino acid sequence having at least, for example, 95% “identity” to a reference amino acid sequence of SEQ ID NO: 2, it is intended that the amino acid sequence of the polypeptide is identical to the reference sequence except that the polypeptide sequence may include up to five amino acid alterations for every 100 amino acids of the reference amino acid sequence of SEQ ID NO: 2. In other words, to obtain a polypeptide having an amino acid sequence at least 95% identical to a reference amino acid sequence, up to 5% of the amino acid residues in the reference sequence may be deleted or substituted with another amino acid, or a number of amino acids up to 5% of the total amino acid residues in the reference sequence may be inserted into the reference sequence. These alterations of the reference sequence may occur at the amino or carboxy terminal positions of the reference amino acid sequence, or anywhere between those terminal positions, interspersed either individually among residues in the reference sequence or in one or more continuous groups within the reference sequence.

The term “biologically active” or “biological activity”, as used herein, refer to a protein having structural, regulatory, biochemical, electrophysiological or cellular functions of a naturally occurring molecule. Likewise, “immunologically active” refers to the capability of the natural, recombinant, or synthetic hASIC1B, or any oligopeptide thereof, to induce a specific immune response in appropriate animals or cells and to bind with specific antibodies.

As used herein, “proton-gated” and “acid-sensing” refer to an increase in cation permeability of a channel molecule induced by an increase in proton ion concentration, also described as increased acidity or lowering of pH.

“Gain of function” refers to hASIC1B derivatives, which show a potentiation of an existing biological activity and/or an acquisition of a novel biological activity. Similarly, “loss of function” refers to hASIC1B derivatives, which show a partial or complete loss of one or more existing biological activities. The expression “dominant-negative” refers to a hASIC1B derivative with a loss of function which, when coexpressed with a fully functional hASIC1B in vivo, for example as a transgene, or in vitro, for example in an assay used to test the specific biological activity (for example “acid-sensing”), will dominate the response and impose the loss of biological activity on all other hASIC1B subunits associated with it. The dominant-negative effect can also manifest itself in conditions where the dominant-negative hASIC1B derivative is coexpressed with other functional ASIC family members, such as but not limited to ASIC1A, ASIC2A or ASIC3, and vice versa where dominant-negative ASIC subunits are co-expressed with functional hASIC1B.

The term “agonist”, as used herein, refers to a molecule which, when bound to hASIC1B, causes a change in hASIC1B which modulates the activity of hASIC1B. Agonists may include proteins, nucleic acids, carbohydrates, or any other molecules which bind to hASIC1B.

The terms “antagonist” or “inhibitor”, as used herein, refer to a molecule which, when bound to hASIC1B, modulates or blocks the biological or immunological activity of hASIC1B. Antagonists and inhibitors may include proteins, nucleic acids, carbohydrates, or any other molecules which bind to hASIC1B.

The term “modulate”, as used herein, refers to a change or an alteration in the biological activity of hASIC1B. Modulation may be an increase or a decrease in protein activity, a change in binding characteristics, or any other change in the biological, functional or immunological properties of hASIC1B.

The term “mimetic”, as used herein, refers to a molecule, the structure of which is developed from knowledge of the structure of hASIC1B or portions thereof and, as such, is able to effect some or all of the actions of ASIC-like molecules.

The term “substantially purified”, as used herein, refers to nucleic or amino acid sequences that are removed from their natural environment, isolated or separated, and are at least 60% free, preferably 75% free, more preferably 90%, even more preferable 95%, and most preferably 99% free from other components with which they are naturally associated.

“Amplification” as used herein refers to the production of additional copies of a nucleic acid sequence and is generally carried out using polymerase chain reaction (PCR) technologies well known in the art (“PCR Primer: a laboratory manual” Dieffenbach C W and Dveksler G S, eds., 1995, CSHL Press, Plainview, N.Y.)

The term “hybridization”, as used herein, refers to any process by which a strand of nucleic acid binds with a complementary strand through base pairing.

The term “hybridization complex”, as used herein, refers to a complex formed between two nucleic acid sequences by virtue of the formation of hydrogen bonds between complementary G and C bases and between complementary A and T bases; these hydrogen bonds may be further stabilized by base stacking interactions. The two complementary nucleic acid sequences hydrogen bond in an antiparallel configuration. A hybridization complex may be formed in solution (e.g., RNAse Protection Assay analysis) or between one nucleic acid sequence present in solution and another nucleic acid sequence immobilized on a solid support (e.g., membranes, filters, chips, pins or glass slides to which cells have been fixed for in situ hybridization).

The terms “complementary” or “complementarity”, as used herein, refer to the natural binding of polynucleotides under permissive salt and temperature conditions by base-pairing. For example, for the sequence “A-G-T” binds to the complementary sequence “T-C-A”. Complementarity between two single-stranded molecules may be “partial”, in which only some of the nucleic acids bind, or it may be complete when total complementarity exists between the single-stranded molecules. The degree of complementarity between nucleic acid strands has significant effects on the efficiency and strength of hybridization between nucleic acid strands. This is of particular importance in amplification reactions, which depend upon binding between nucleic acids strands.

The term “homology”, as used herein, refers to the degree of complementarity of sequences or probes. There may be partial homology or complete homology (i.e., identity). A partially complementary sequence is one that at least partially inhibits an identical sequence from hybridizing to a target nucleic acid; it is referred to using the functional term “substantially homologous.” The inhibition of hybridization of the completely complementary sequence to the target sequence may be examined using a hybridization assay (Southern or northern blot, solution hybridization and the like) under conditions of low stringency. A substantially homologous sequence or probe will compete for and inhibit the binding (i.e., the hybridization) of a completely homologous sequence or probe to the target sequence under conditions of low stringency. This is not to say that conditions of low stringency are such that non-specific binding is permitted; low stringency conditions require that the binding of two sequences to one another be a specific (i.e., selective) interaction. The absence of non-specific binding may be tested by the use of a second target sequence which lacks even a partial degree of complementarity (e.g., less than about 30% identity); in the absence of non-specific binding, the probe will not hybridize to the second non-complementary target sequence.

As known in the art, numerous equivalent conditions may be employed to comprise either low or high stringency conditions. Factors such as the length and nature of the sequence (DNA, RNA, base composition), nature of the target (DNA, RNA, base composition, presence in solution or immobilization, etc.), and the concentration of the salts and other components (e.g., the presence or absence of formamide, dextran sulfate and/or polyethylene glycol) are considered and the hybridization solution may be varied to generate conditions of either low or high stringency different from, but equivalent to, the conditions listed above.

The term “stringent conditions”, as used herein, is the “stringency” which occurs within a range from about Tm-5° C. (5° C. below the melting temperature (Tm) of the probe) to about 20° C. to 25° C. below Tm. As will be understood by those of skill in the art, the stringency of hybridization may be altered in order to identify or detect identical or related polynucleotide sequences.

The term “antisense”, as used herein, refers to nucleotide sequences which are complementary to a specific DNA or RNA sequence. The term “antisense strand” is used in reference to a nucleic acid strand that is complementary to the “sense” strand. Antisense molecules may be produced by any method, including synthesis by ligating the gene(s) of interest in a reverse orientation to a viral promoter, which permits the synthesis of a complementary strand. Once introduced into a cell, this transcribed strand combines with natural sequences produced by the cell to form duplexes. These duplexes then block either the further transcription or translation. In this manner, mutant phenotypes may be generated. The designation “negative” is sometimes used in reference to the antisense strand, and “positive” is sometimes used in reference to the sense strand.

The term “portion”, as used herein, with regard to a protein (as in “a portion of a given protein”) refers to fragments of that protein. The fragments may range in size from four amino acid residues to the entire amino acid sequence minus one amino acid. Thus, a protein “comprising at least a portion of the amino acid sequence of SEQ ID NO:2” encompasses the full-length human hASIC1B and fragments thereof.

“Transformation”, as defined herein, describes a process by which exogenous DNA enters and changes a recipient cell. It may occur under natural or artificial conditions using various methods well known in the art. Transformation may rely on any known method for the insertion of foreign nucleic acid sequences into a prokaryotic or eukaryotic host cell. The method is selected based on the host cell being transformed and may include, but is not limited to, viral infection, electroporation, lipofection, and particle bombardment. Such “transformed” cells include stably transformed cells in which the inserted DNA is capable of replication either as an autonomously replicating plasmid or as part of the host chromosome. They also include cells which transiently express the inserted DNA or RNA for limited periods of time.

The term “antigenic determinant”, as used herein, refers to that portion of a molecule that makes contact with a particular antibody (i.e., an epitope). When a protein or fragment of a protein is used to immunize a host animal, numerous regions of the protein may induce the production of antibodies which bind specifically to a given region or three-dimensional structure on the protein; these regions or structures are referred to as antigenic determinants. An antigenic determinant may compete with the intact antigen (i.e., the immunogen used to elicit the immune response) for binding to an antibody.

The terms “specific binding” or “specifically binding”, as used herein, in reference to the interaction of an antibody and a protein or peptide, means that the interaction is dependent upon the presence of a particular structure (i.e., the antigenic determinant or epitope) on the protein; in other words, the antibody is recognizing and binding to a specific protein structure rather than to proteins in general. For example, if an antibody is specific for epitope “A”, the presence of a protein containing epitope A (or free, unlabeled A) in a reaction containing labeled “A” and the antibody will reduce the amount of labeled A bound to the antibody.

The term “sample”, as used herein, is used in its broadest sense. A biological sample suspected of containing nucleic acid encoding hASIC1B or fragments thereof may comprise a cell, chromosomes isolated from a cell (e.g., a spread of metaphase chromosomes), genomic DNA (in solution or bound to a solid support such as for Southern analysis), RNA (in solution or bound to a solid support such as for northern analysis), cDNA (in solution or bound to a solid support), an extract from cells or a tissue, and the like.

The term “correlates with expression of a polynucleotide”, as used herein, indicates that the detection by northern analysis and/or RT-PCR of the presence of ribonucleic acid that is related to SEQ ID NO:1 is indicative of the presence of mRNA encoding hASIC1B in a sample and thereby correlates with expression of the transcript encoding the protein.

“Alterations” in the polynucleotide of SEQ ID NO:1, as used herein, comprise any alteration in the sequence of polynucleotides encoding hASIC1B including deletions, insertions, and point mutations that may be detected using hybridization assays. Included within this definition is the detection of alterations to the genomic DNA sequence which encodes hASIC1B (e.g., by alterations in the pattern of restriction fragment length polymorphisms capable of hybridizing to SEQ ID NO:1), the inability of a selected fragment of SEQ ID NO:1 to hybridize to a sample of genomic DNA (e.g., using allele-specific oligonucleotide probes), and improper or unexpected hybridization, such as hybridization to a locus other than the normal chromosomal locus for the polynucleotide sequence encoding hASIC1B (e.g., using fluorescent in situ hybridization (FISH) to metaphase chromosomes spreads).

As used herein, the term “antibody” refers to intact molecules as well as fragments thereof, such as Fa, F(ab′)₂, and Fv, which are capable of binding the epitopic determinant. Antibodies that bind hASIC1B polypeptides can be prepared using intact polypeptides or fragments containing small peptides of interest as the immunizing antigen. The polypeptide or peptide used to immunize an animal can be derived from translated RNA or synthesized chemically, and can be conjugated to a carrier protein, if desired. Commonly used carriers that are chemically coupled to peptides include bovine serum albumin and thyroglobulin. The coupled peptide is then used to immunize the animal (e.g., a mouse, a rat or a rabbit). These methods are well described in the literature: e.g. “Antobodies: A Laboratory Manual”, Harlow E and Lane D, eds., 1998, CSHL Press, Plainview, N.Y.)

The term “humanized antibody”, as used herein, refers to antibody molecules in which amino acids have been replaced in the non-antigen binding regions in order to more closely resemble a human antibody, while still retaining the original binding ability.

Disclosure of the Invention

The present invention is based on the discovery of a novel human Acid Sensing Ion Channel protein, hASIC1B, the polynucleotides encoding hASIC1B, and the use of these compositions for diagnosis, prevention, or treatment of disease.

Nucleic acids encoding the human hASIC1B of the present invention were first identified by a web-based virtual screening of the GenBank database. The TblastN search was performed using the rat ASIC1B amino acid sequence as the input query sequence. A human genomic clone AC025154 (GenBank) was thus identified. The identified sequences allowed the subsequent synthesis of specific oligonudeotide primers, which enabled the isolation of the full length hASIC1B nucleic acid molecule of SEQ ID NO: 1 by RT-PCR using reverse transcribed cDNA from mRNA isolated from human trigeminal ganglia.

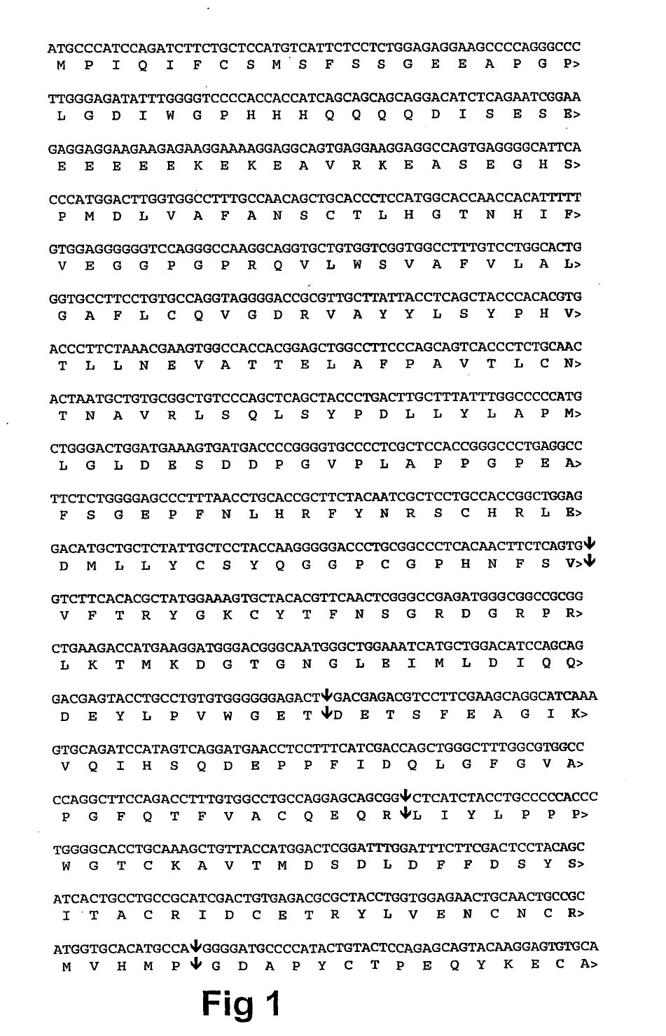

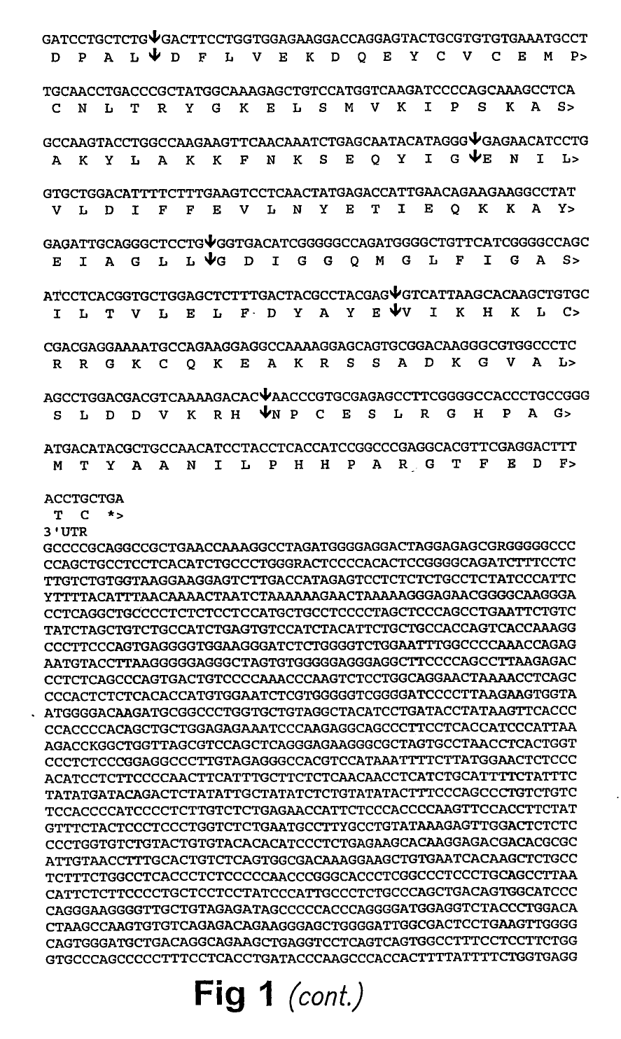

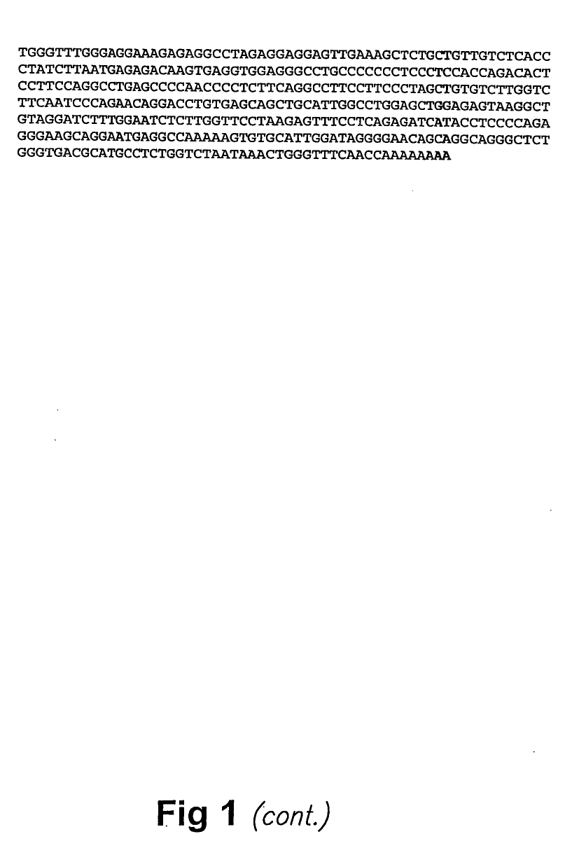

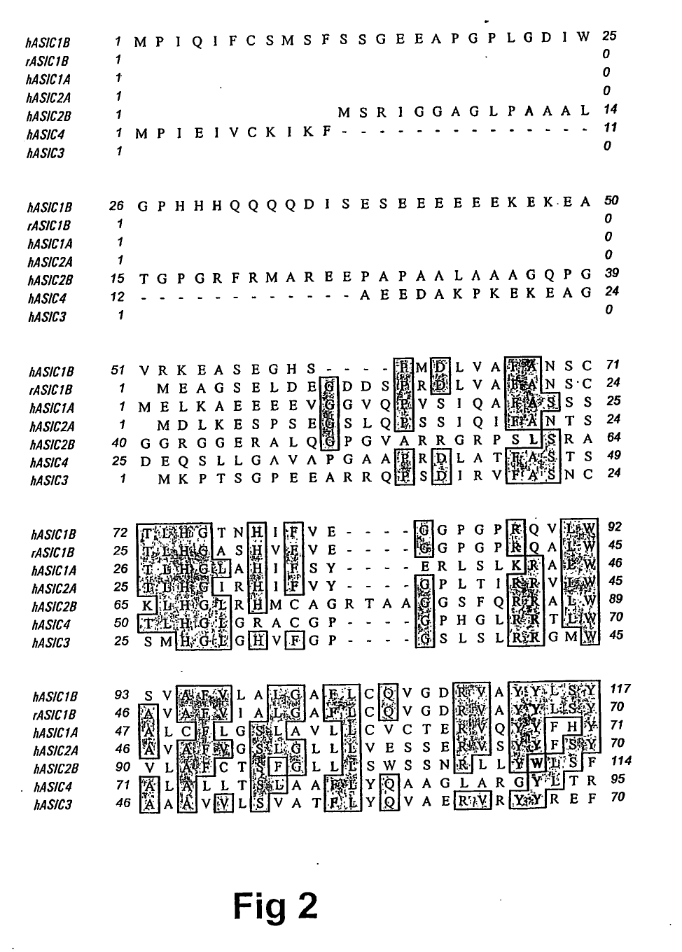

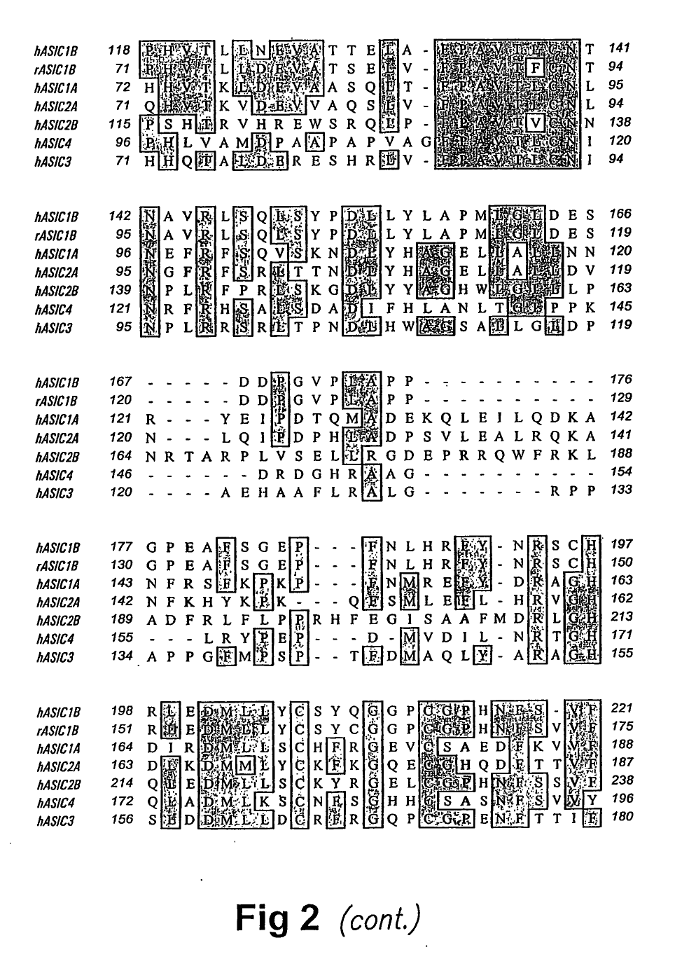

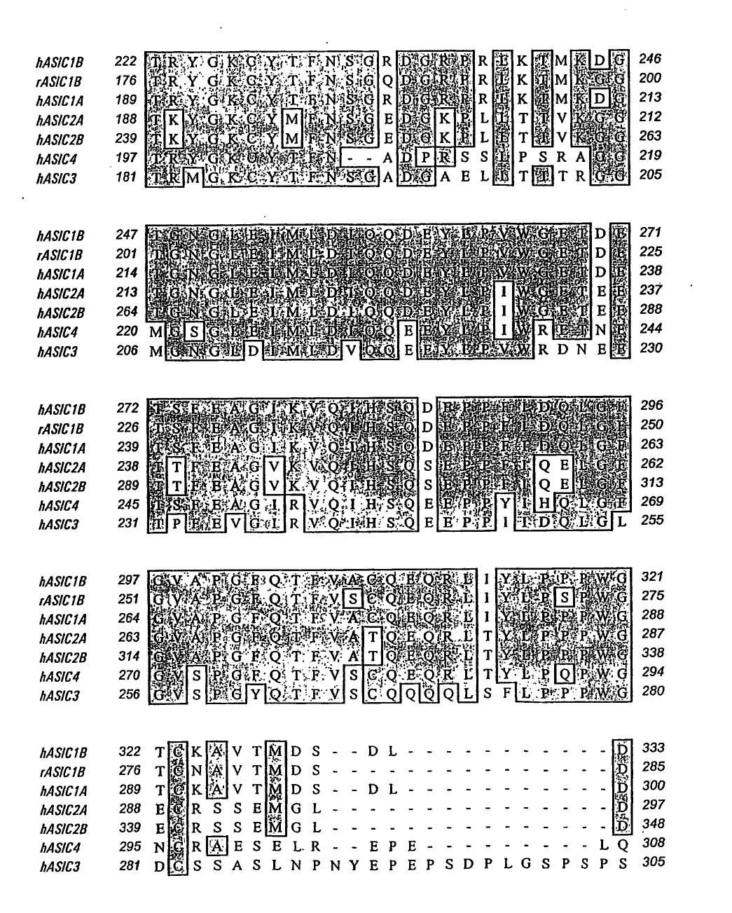

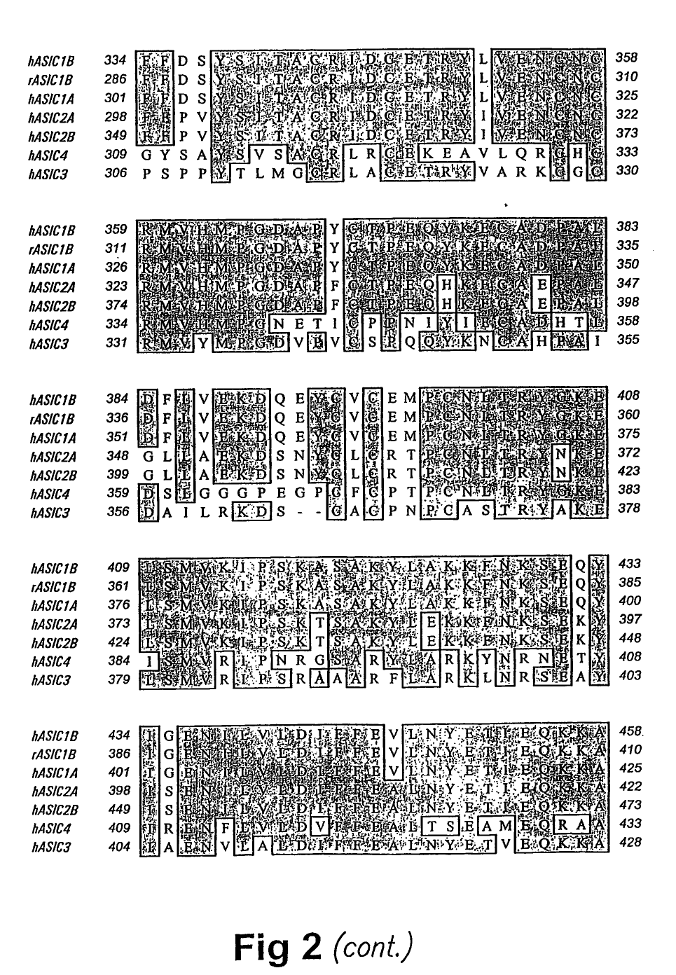

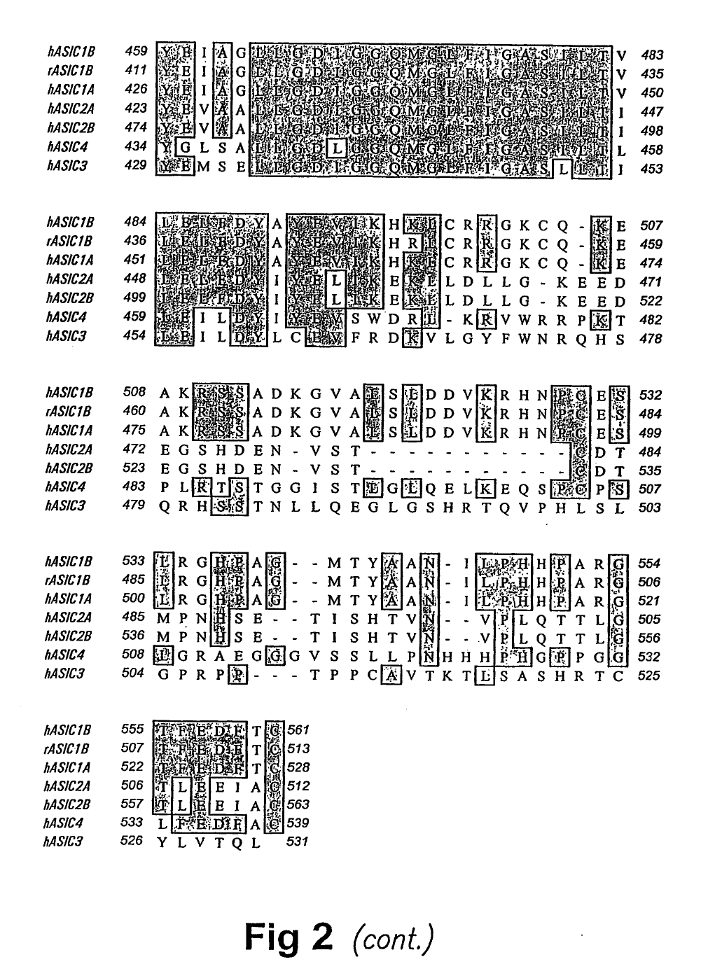

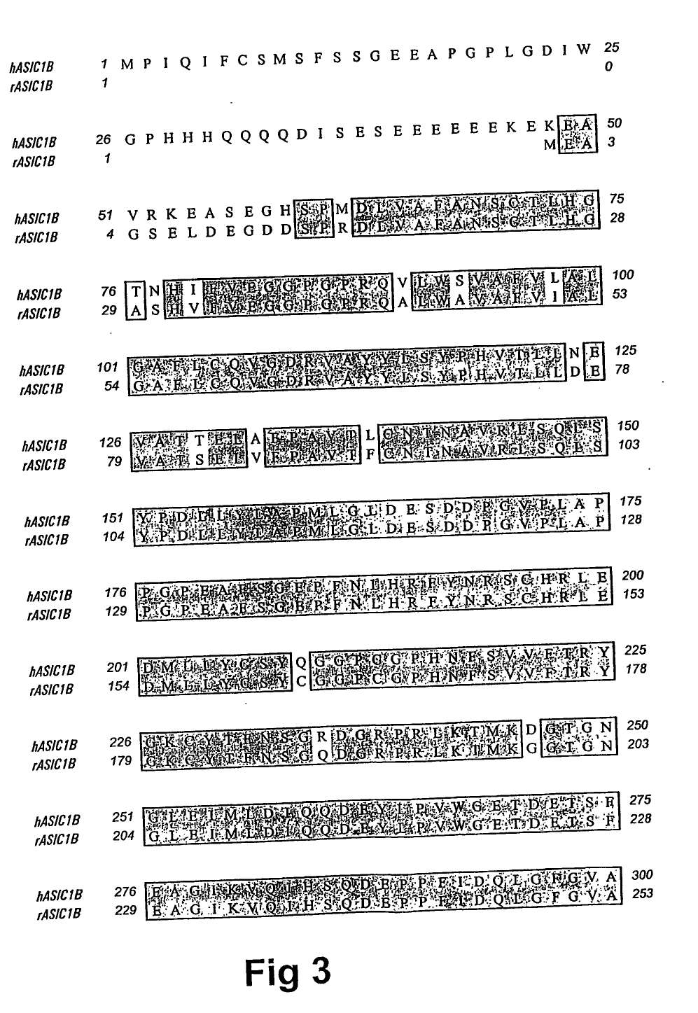

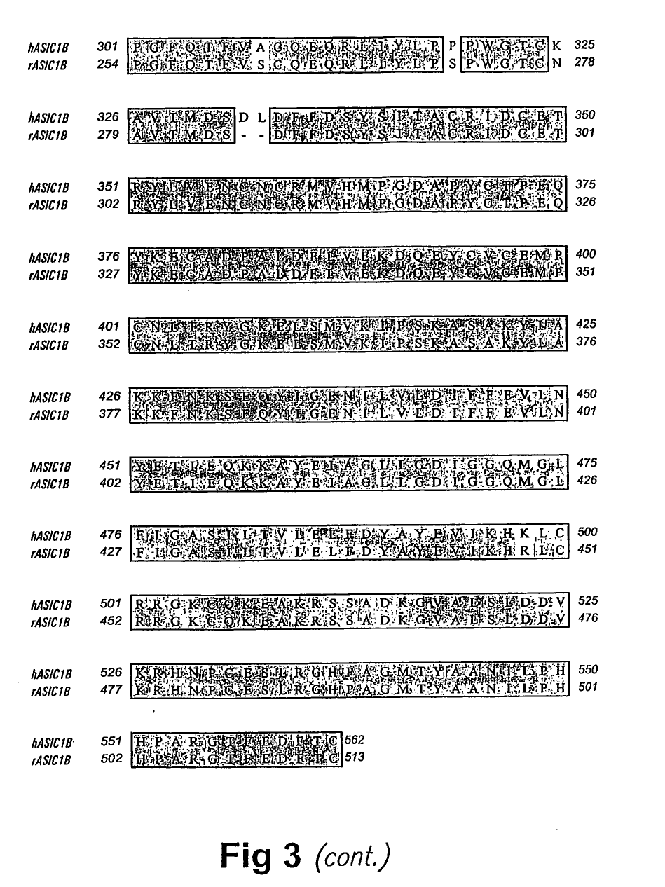

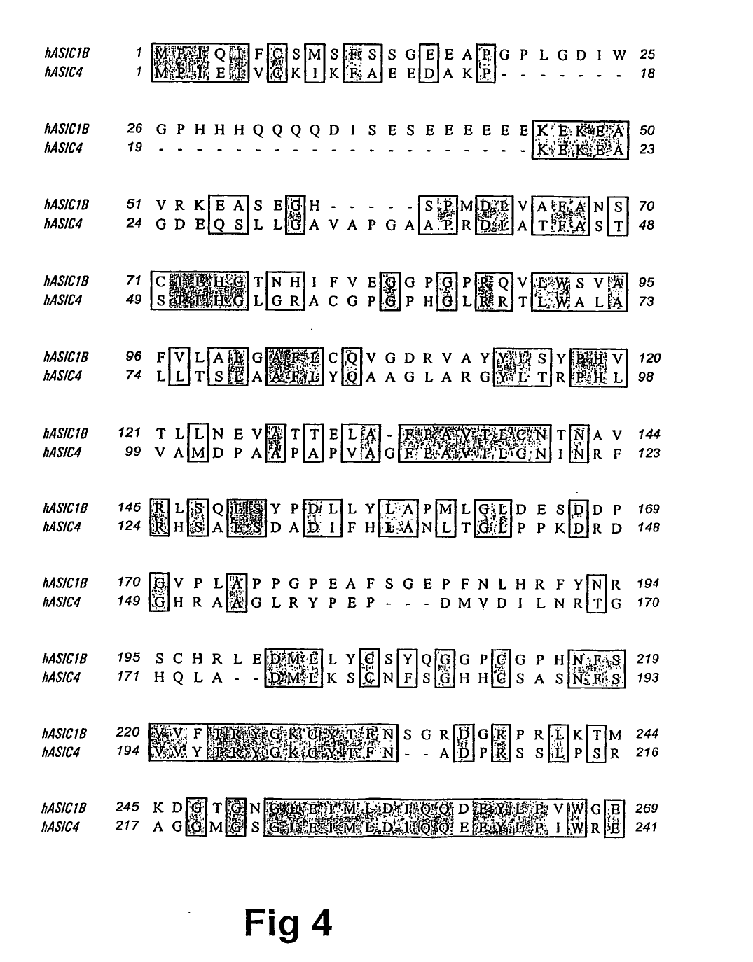

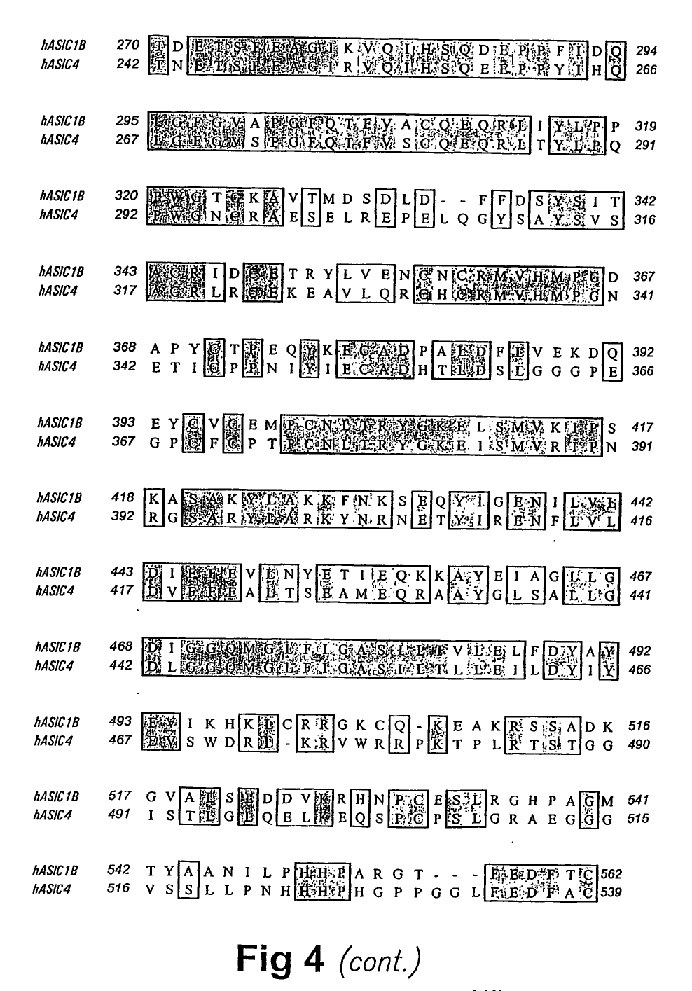

In one embodiment, the invention encompasses the novel human proton-gated ion channel, a polypeptide comprising the amino acid sequence of SEQ ID NO: 2, as shown in FIG. 1. hASIC1B is 562 amino acids in length and has two potential hydrophobic transmembrane domains. As shown in FIG. 2, hASIC1B has chemical and structural homology with other members of the ASIC gene-family. In particular, some motifs or stretches of amino acids are completely conserved in all ASIC subunits identified to date. Human hASIC1B shows the highest identity with the published rat ASIC beta subunit (GenBank Accession No.), but is extended by 47 aa on the N-terminal side. Northern blot and RT-PCR analysis reveals that hASIC1B is expressed in the central and peripheral nervous system, with a strong enrichment in sensory ganglia.

The invention also encompasses hASIC1B variants. A preferred hASIC1B variant is one having at least 80%, and more preferably 90%, amino acid sequence identity to the hASIC1B amino acid sequence (SEQ ID NO: 2). A most preferred hASIC1B variant is one having at least 95% amino acid sequence identity to SEQ ID NO: 2, while those with 97-99% amino acid sequence identity are most highly preferred.

The invention also encompasses polynucleotides, which encode hASIC1B polypeptides. Accordingly, any nucleic acid sequence, which encodes the amino acid sequence of hASIC1B can be used to generate recombinant molecules which express hASIC1B. In a particular embodiment, the invention encompasses the polynucleotide comprising the nucleic acid of SEQ ID NO: 1 as shown in FIG. 1.

It will be appreciated by those skilled in the art that as a result of the degeneracy of the genetic code, a multitude of nucleotide sequences encoding hASIC1B, some bearing minimal homology to the nucleotide sequences of any known and naturally occurring gene, may be produced. Thus, the invention contemplates each and every possible variation of nucleotide sequence that could be made by selecting combinations based on possible codon choices. These combinations are made in accordance with the standard triplet genetic code as applied to the nucleotide sequence of naturally occurring hASIC1B, and all such variations are to be considered as being specifically disclosed.

Although nucleotide sequences which encode hASIC1B and its variants are preferably capable of hybridizing to the nucleotide sequence of the naturally occurring hASIC1B under appropriately selected conditions of stringency, it may be advantageous to produce nucleotide sequences encoding hASIC1B or its derivatives possessing a substantially different codon usage. Codons may be selected to increase the rate at which expression of the peptide occurs in a particular prokaryotic or eukaryotic expression host in accordance with the frequency with which particular codons are utilized by the host. Other reasons for substantially altering the nucleotide sequence encoding hASIC1B and its derivatives without altering the encoded amino acid sequences include the production of RNA transcripts having more desirable properties, such as a greater half-life, than transcripts produced from the naturally occurring sequence.

The invention also encompasses production of a DNA sequence, or portions thereof, which encode hASIC1B and its derivatives, entirely by synthetic chemistry. After production, the synthetic gene may be inserted into any of the many available DNA vectors and cell systems using reagents that are well known in the art at the time of the filing of this application. Moreover, synthetic chemistry may be used to introduce mutations into a sequence encoding hASIC1B or any portion thereof.

Also encompassed by the invention are polynucleotide sequences that are capable of hybridizing to the claimed nucleotide sequences, and in particular, those shown in SEQ ID NO: 1, under various conditions of stringency. Hybridization conditions are based on the melting temperature (Tm) of the nucleic acid binding complex or probe, as taught in Berger and Kimmel (Meth Enzymol 1987: 152), and may be used at a defined stringency.

Altered nucleic acid sequences encoding hASIC1B which are encompassed by the invention include deletions, insertions, or substitutions of different nucleotides resulting in a polynucleotide that encodes the same or a functionally equivalent hASIC1B. The encoded protein may also contain deletions, insertions, or substitutions of amino acid residues, which result in a functionally equivalent hASIC1B. Also encompassed by the invention are altered nucleic acid sequences, including deletions, insertions or substitutions, which result in a polynucleotide that encodes an hASIC1B polypeptide with increased or novel biological activity (“gain of function”), or an hASIC1B polypeptide with decreased or suppressed biological activity (“Loss of function” or “Dominant-negative”). The encoded protein may also contain deletions, insertions, or substitutions of amino acid residues, which result in a functionally divergent hASIC1B, as described herein above. Deliberate amino acid substitutions may be made on the basis of similarity in polarity, charge, solubility, hydrophobicity, hydrophilicity, and/or the amphipathic nature of the residues as long as the biological activity of hASIC1B is retained. For example, negatively charged amino acids may include aspartic acid and glutamic acid; positively charged amino acids may include lysine and arginine; and amino acids with uncharged polar head groups having similar hydrophilicity values may include leucine, isoleucine, and valine; glycine and alanine; asparagine and glutamine; serine and threonine; phenylalanine and tyrosine.

Also included within the scope of the present invention are alleles of the gene encoding hASIC1B. As used herein, an “allele” or “allelic sequence” is an alternative form of the gene, which may result from at least one mutation in the nucleic acid sequence. Alleles may result in altered mRNAs or polypeptides whose structure or function may or may not be altered. Any given gene may have none, one, or many allelic forms. Common mutational changes, which give rise to alleles, are generally ascribed to natural deletions, additions, or substitutions of nucleotides. Each of these types of changes may occur alone, or in combination with the others, one or more times in a given sequence.

Methods for DNA sequencing, which are well known and generally available in the art, may be used to practice any embodiments of the invention. The methods may employ such enzymes as the Klenow fragment of DNA polymerase I, Sequenase II (US Biochemical Corp, Cleveland, Ohio), Taq polymerase (Perkin Elmer), thermostable T7 polymerase (Amersham, Chicago, Ill.), or combinations of recombinant polymerases and proofreading exonucleases such as the ELONGASE Amplification System marketed by Gibco BRL (Gaithersburg, Md.). Preferably, the process is automated with machines such as the Hamilton Micro Lab 2200 (Hamilton, Reno, Nev.), Peltier Thermal Cycler (PTC200; M.J. Research, Watertown, Mass.) and the ABI 377 DNA sequencers (Perkin Elmer), to name a few.

The polynucleotide sequence encoding hASIC1B may be extended utilizing a partial nucleotide sequence and employing various methods known in the art to detect upstream sequences such as promoters and regulatory elements. For example, one method which may be employed, “restriction-site” PCR, uses universal primers to retrieve unknown sequences adjacent to a known locus (Sarkar et al., PCR Meth Applic 1993; 2:318-322). In particular, genomic DNA is first amplified in the presence of primer to linker sequence and a primer specific to the known region. The amplified sequences are then subjected to a second round of PCR with the same linker primer and another specific primer internal to the first one. Products of each round of PCR are transcribed with an appropriate RNA polymerase and sequenced using reverse transcriptase.

Inverse PCR may also be used to amplify or extend sequences using divergent primers based on a known region (Triglia et al., Nuc. Acid Res. 1988; 16: 8186). The primers may be designed using GeneWorks 2.5.1 or MacVector 6.0.1 (Oxford Molecular Group, Cambridge, UK), or also OLIGO 4.06 Primer Analysis software (National Biosciences Inc., Plymouth, Minn.), or any other appropriate program, to be 22-30 nucleotides in length, to have a GC content of 50% or more, and to anneal to the target sequence at temperatures about 68-72° C. The method uses several restriction enzymes to generate a suitable fragment in the known region of a gene. The fragment is then circularized by intramolecular ligation and used as a PCR template.

Another method which may be used is capture PCR which involves PCR amplification of DNA fragments adjacent to a known sequence in human and yeast artificial chromosome DNA (Lagerstrom et al., PCR Meth Applic 1991; 1: 111). In this method, multiple restriction enzyme digestions and ligations may also be used to place an engineered double-stranded sequence into an unknown portion of the DNA molecule before performing PCR.

Another method which may be used to retrieve unknown sequences is that of Parker et al. (Nuc Acid Res. 1991; 19: 3055). Additionally, one may use PCR, nested primers, and PromoterFinder™ libraries to walk in genomic DNA (Clontech, Palo Alto, Calif.). This process avoids the need to screen libraries and may be is useful in finding intron/exon junctions.

When screening for full-length cDNAs, it is preferable to use libraries that have been size-selected to include larger cDNAs. Also, random-primed libraries are preferable in that they will contain more sequences which contain the 5′ regions of genes. Use of a randomly primed library may be especially preferable for situations in which an oligo d(T) library does not yield a full-length cDNA. Genomic libraries may be useful for extension of sequence into the 5′ and 3′ non-translated regulatory regions.

Capillary electrophoresis systems which are commercially available may be used to analyze the size or confirm the nucleotide sequence of sequencing or PCR products. In particular, capillary sequencing may employ flowable polymers for electrophoretic separation, four different fluorescent dyes (one for each nucleotide) which are laser activated, and detection of the emitted wavelengths by a charge coupled devise camera. Output/light intensity may be converted to electrical signal using appropriate software (e.g. Genotyper™ and Sequence Navigator™, Perkin Elmer) and the entire process from loading of samples to computer analysis and electronic data display may be computer controlled. Capillary electrophoresis is especially preferable for the sequencing of small pieces of DNA which might be present in limited amounts in a particular sample.

In another embodiment of the invention, polynucleotide sequences or fragments thereof which encode hASIC1B, or fusion proteins or functional equivalents thereof, may be used in recombinant DNA molecules to direct expression of hASIC1B. Due to the inherent degeneracy of the genetic code, other DNA sequences which encode substantially the same or a functionally equivalent amino acid sequence may be produced and these sequences may be used to clone and express hASIC1B.

As will be understood by those of skill in the art, it may be advantageous to produce hASIC1B-encoding nucleotide sequences possessing non-naturally occurring codons. For example, codons preferred by a particular prokaryotic or eukaryotic host can be selected to increase the rate of protein expression or to produce a recombinant RNA transcript having desirable properties, such as a half-life which is longer than that of a transcript generated from the naturally occurring sequence.

The nucleotide sequences of the present invention can be engineered using methods generally known in the art in order to alter the hASIC1B coding sequence for a variety of reasons, including but not limited to, alterations which modify the cloning, processing, and/or expression of the gene product. DNA shuffling by random fragmentation and PCR reassembly of gene fragments and synthetic oligonucleotides may be used to engineer the nucleotide sequence. For example, site-directed mutagenesis may be used to insert new restriction sites, alter glycosylation patterns, to change codon preference, to produce splice variants, or other mutations, and so forth. Alternatively, the nucleotide sequences can be engineered to generate chimeric ASIC channels, where portions of the hASIC1B channel are substituted with equivalent portions of other ASIC subunits, for example the ASIC1A or ASIC 2A.

In another embodiment of the invention, a natural, modified, or recombinant polynucleotide encoding hASIC1B may be ligated to a heterologous sequence to encode a fusion protein. For example, to screen peptide libraries for inhibitors of hASIC1B activity, it may be useful to encode a chimeric hASIC1B protein that can be recognized by a commercially available antibody. A fusion protein may also be engineered to contain a cleavage site located between an hASIC1B encoding sequence and the heterologous protein sequence, so that hASIC1B may be cleaved and purified away from the heterologous moiety.

In another embodiment, the coding sequence of hASIC1B may be synthesized, in whole or in part, using chemical methods well known in the art (see Caruthers et al., Nuc. Acids Res. Symp. Ser. 1980; 215-23; Horn et al., Nuc. Acids Res. Symp. Ser. 1980; 225-232). Alternatively, the protein itself may be produced using chemical methods to synthesize the hASIC1B amino acid sequence, or a portion thereof. For example, peptide synthesis can be performed using various solid-phase techniques (Roberge et al., Science 1995; 269: 202) and automated synthesis may be achieved, for example, using the ABI 431A Peptide Synthesizer (Perkin Elmer).

The newly synthesized peptide may be substantially purified by preparative high performance liquid chromatography e.g., Creighton T. (1983) “Proteins, Structures and Molecular Principles”, W. H. Freeman & Co., New York, N.Y.). The composition of the synthetic peptides may be confirmed by amino acid analysis or sequencing (e.g., the Edman degradation procedure; Creighton T (1983), supra). Additionally, the amino acid sequence of hASIC1B, or any part thereof, may be altered during direct synthesis and/or combined using chemical methods with sequences from other proteins, or any part thereof, to produce a variant polypeptide.

In order to express a biologically active hASIC1B, the nucleotide sequence encoding hASIC1B or functional equivalents may be inserted into an appropriate expression vector, i.e., a vector, which contains the necessary elements for the transcription and translation of the inserted coding sequence.

Methods which are well known to those skilled in the art may be used to construct expression vectors containing a hASIC1B coding sequence and appropriate transcriptional and translational control elements. These methods include in vitro recombinant DNA techniques, synthetic techniques, and in vivo genetic recombination. Such techniques are described in “Molecular Cloning: A Laboratory Manual”, Sambrook J, Ed., CSHL Press, 1989, Cold Spring Harbor, N.Y., and “Current Protocols in Molecular Biology”, Ausubel et al., John Wiley & Sons, 1989, New York, N.Y.

A variety of expression vector/host systems may be utilized to contain and express a hASIC1B coding sequence. These include, but are not limited to, microorganisms such as bacteria transformed with recombinant bacteriophage, plasmid, or cosmid DNA expression vectors; yeast transformed with yeast expression vectors; insect cell systems infected with virus expression vectors (e.g., baculovirus); plant cell systems transformed with virus expression vectors (e.g., cauliflower mosaic virus, CaMV; tobacco mosaic virus, TMV) or with bacterial expression vectors (e.g., Ti or pBR322 plasmids); or animal cell systems.

The “control elements” or “regulatory sequences” are those non-translated regions of the vector—enhancers, promoters, 5′ and 3′ untranslated regions—which interact with host cellular proteins to carry out transcription and translation. Such elements may vary in their strength and specificity. Depending on the vector system and host utilized, any number of suitable transcription and translation elements, including constitutive and inducible promoters, may be used. For example, when cloning in bacterial systems, inducible promoters such as the hybrid lacZ promoter of the Bluescript® phagemid (Stratagene, La Jolla, Calif.) or pSport1™ plasmid (Gibco BRL) and ptrp-lac hybrids, and the like may be used. Other preferred prokaryotic vectors include but are not limited to pQE-9, pQE60, pQE70 (Quiagen), pNH8A, pNH16a, pNH18a, pNH46A (Stratagene) ptrc99a, pKK223-3, pKK233-3, pDR540, pRIT5 (Pharmacia). The baculovirus polyhedrin promoter may be used in insect cells. Promoters or enhancers derived from the genomes of plant cells (e.g., heat shock, RUBISCO; and storage protein genes) or from plant viruses (e.g., viral promoters or leader sequences) may be cloned into the vector. In mammalian cell systems, promoters from mammalian genes or from mammalian viruses are preferable. If it is necessary to generate a cell line that contains multiple copies of the sequence encoding hASIC1B, vectors based on SV40 or EBV may be used with an appropriate selectable marker.

In bacterial systems, a number of expression vectors may be selected depending upon the use intended for hASIC1B. For example, when large quantities of hASIC1B are needed for the induction of antibodies, vectors, which direct high level expression of fusion proteins that are readily purified, may be used. Such vectors include, but are not limited to, the multifunctional E. coli cloning and expression vectors such as Bluescript® (Stratagene, La Jolla, Calif.), in which the sequence encoding hASIC1B may be ligated into the vector in frame with sequences for the amino-terminal Methionine and the subsequent 7 residues of β-galactosidase so that a hybrid protein is produced; pIN vectors (Van Heeke and Schuster, J. Biol. Chem. 1989; 264: 5503); and the like; pGEX vectors (Promega, Madison, Wis.) may also be used to express foreign polypeptides as fusion proteins with glutathione S-transferase (GST). In general, such fusion proteins are soluble and can easily be purified from lysed cells by adsorption to glutathione-agarose beads followed by elution in the presence of free glutathione. Proteins made in such systems may be designed to include heparin, thrombin, or factor XA protease cleavage sites so that the cloned polypeptide of interest can be released from the GST moiety at will.

In addition to bacteria, eucaryotic microbes, such as yeast, may also be used as hosts. Laboratory strains of Saccharomyces cerevisiae, Baker's yeast, are most used although a number of other strains or species are commonly available. Vectors employing, for example, the 2 μ origin of replication of Broach et al. (Meth Enzymol 1983; 101: 307), or other yeast compatible origins of replication (see, for example, Stinchcomb et al. Nature 1979: 282; 39, Tschumper et al., Gene 1980: 10; 157, Clarke et al., Meth Enzymol 1983; 101: 300) may be used. Control sequences for yeast vectors include promoters for the synthesis of glycolytic enzymes (Hess et al. J Adv Enzyme Reg 1968; 7: 149; Holland et al., Biochemistry 1978; 17: 4900). Additional promoters known in the art include the promoter for 3-phosphoglycerate kinase (Hitzeman et al., J Biol Chem 1980; 255: 2073), alcohol oxidase, and PGH. Other promoters, which have the additional advantage of transcription controlled by growth conditions and/or genetic background are the promoter regions for alcohol dehydrogenase 2, isocytochrome C, acid phosphatase, degradative enzymes associated with nitrogen metabolism, the alpha-factor system and enzymes responsible for maltose and galactose utilization. It is also believed terminator sequences are desirable at the 3′ end of the coding sequences. Such terminators are found in the 3′ untranslated region following the coding sequences in yeast-derived genes. For reviews, see “Current Protocols in Molecular Biology”, Ausubel et al., John Wiley & Sons, 1989, New York, N.Y. and Grant et al., Meth Enzymol. 1987; 153: 516.

In cases where plant expression vectors are used, the expression of a sequence encoding hASIC1B may be driven by any of a number of promoters. For example, viral promoters such as the 35S and 19S promoters of CaMV may be used alone or in combination with the omega leader sequence from TMV (Takamatsu et al., EMBO J. 1987; 6: 307; Brisson et al., Nature 1984; 310: 511). Alternatively, plant promoters such as the small subunit of RUBISCO or heat shock promoters may be used (Coruzzi et al., EMBO J 1984; 3: 1671; Broglie et al., Science 1984; 224: 838; Winter et al., Results Probl. Cell Differ 1991; 17: 85). These constructs can be introduced into plant cells by direct DNA transformation or pathogen-mediated transfection. Such techniques are described in a number of generally available reviews (see, for example, Hobbs S or Murry L E in “McGraw Hill Yearbook of Science and Technology” McGraw Hill, 1992, New York, N.Y.; pp. 191-196 or Weissbach and Weissbach in “Methods for Plant Molecular Biology”, Academic Press, 1988, New York, N.Y.; pp. 421-463).

An insect system may also be used to express hASIC1B. For example, in one such system, Autographa californica nuclear polyhedrosis virus (AcNPV) is used as a vector to express foreign genes in Spodoptera frugiperda cells or in Trichoplusia larvae. The sequence encoding hASIC1B may be cloned into a nonessential region of the virus, such as the polyhedrin gene, and placed under control of the polyhedrin promoter. Successful insertion of hASIC1B will render the polyhedrin gene inactive and produce recombinant virus lacking coat protein. The recombinant viruses may then be used to infect, for example, S. frugiperda cells or Trichoplusia larvae in which hASIC1B may be expressed (Smith et al., J Virol 1983; 46: 584; Engelhard et al., Proc Natl Acad Sci 1994; 91: 3224).

In mammalian host cells, a number of viral-based expression systems may be utilized. In cases where an adenovirus is used as an expression vector, a sequence encoding hASIC1B may be ligated into an adenovirus transcription/translation complex consisting of the late promoter and tripartite leader sequence. Insertion in a non-essential E1 or E3 region of the viral genome may be used to obtain a viable virus, which is capable of expressing hASIC1B in infected host cells (Logan and Shenk, Proc Natl Acad Sci 1984; 81: 3655). In addition, transcription enhancers, such as the Rous sarcoma virus (RSV) enhancer, may be used to increase expression in mammalian host cells.

Specific initiation signals may also be used to achieve more efficient translation of a sequence encoding hASIC1B. Such signals include the ATG initiation codon and adjacent sequences. In cases where sequences encoding hASIC1B, its initiation codon, and upstream sequences are inserted into the appropriate expression vector, no additional transcriptional or translational control signals may be needed. However, in cases where only coding sequence, or a portion thereof, is inserted, exogenous translational control signals including the ATG initiation codon should be provided. Furthermore, the initiation codon should be in the correct reading frame to ensure the correct translation of the entire insert. Exogenous translational elements and initiation codons may be of various origins, both natural and synthetic. The efficiency of expression may be enhanced by the inclusion of enhancers which are appropriate for the particular cell system which is used, such as those described in the literature (Scharf et al., Results Probl Cell Differ 1994; 20: 125; Bittner et al., Meth Enzymol 1987; 153: 516).

In addition, a host cell strain may be chosen for its ability to modulate the expression of the inserted sequences or to process the expressed protein in the desired fashion. Such modifications of the polypeptide include, but are not limited to, acetylation, carboxylation, glycosylation, phosphorylation, lipidation, and acylation. Post-translational processing which cleaves a “prepro” form of the protein may also be used to facilitate correct insertion, folding and/or function. Different host cells such as CHO, HeLa, MDCK, HEK293, WI38, and COS, which have specific cellular machinery and characteristic mechanisms for such post-translational activities, may be chosen to ensure the correct modification and processing of the foreign protein.

In a preferred expression system, cDNA species are injected directly into Xenopus oocyte nuclei thereby allowing for in vitro translation forming a functional proton-gated channel capable of demonstrating functional characteristics of native proton-gated channels including ion selectivity, gating-kinetics, ligand preferences, and sensitivity to pharmacological agents such as amiloride for a model assay which mimics in vivo characteristics.

For long-term, high-yield production of recombinant proteins, stable expression is preferred. For example, cell lines, which stably express hASIC1B, may be transformed using expression vectors which may contain viral origins of replication and/or endogenous expression elements and a selectable marker gene on the same or separate vector. Following the introduction of the vector, cells may be allowed to grow for 1-2 days in an enriched media before they are switched to selective media. The purpose of the selectable marker is to confer resistance to selection, and its presence allows growth and recovery of cells, which successfully express the introduced sequences. Resistant clones of stably transformed cells may be proliferated using tissue culture techniques appropriate to the cell type.

Any number of selection systems may be used to recover transformed cell lines. These include, but are not limited to, the herpes simplex virus thymidine kinase (Wigler et al., Cell 1977; 11: 223) and adenine phospho-ribosyltransferase (Lowy et al., Cell 1980; 22: 817) genes which can be employed in tk± or aprt±cells, respectively. Also, antimetabolite, antibiotic, or herbicide resistance can be used as the basis for selection; for example, dhfr, which confers resistance to methotrexate (Wigler et al., Proc Natl Acad Sci 1980; 77: 3567); npt, which confers resistance to the aminoglycosides neomycin and G418 (Colbere-Garapin et al., J Mol Biol 1981; 150: 1) and als or pat, which confer resistance to chlorsulfuron and phosphinotricin acetyltransferase, respectively (Murry L E, supra). Additional selectable genes have been described, for example, trpB, which allows cells to utilize indole in place of tryptophan, or hisD, which allows cells to utilize histinol in place of histidine (Hartman and Mulligan, Proc Natl Acad Sci 1988; 85: 8047). Recently, the use of visible markers has gained popularity with such markers as anthocyanins, β-glucuronidase and its substrate GUS, and luciferase and its substrate luciferin, being widely used not only to identify transformants, but also to quantify the amount of transient or stable protein expression attributable to a specific vector system (Rhodes et al., Methods Mol Biol 1995; 55: 121).

Although the presence/absence of marker gene expression suggests that the gene of interest is also present, its presence and expression may need to be confirmed. For example, if the sequence encoding hASIC1B is inserted within a marker gene sequence, recombinant cells containing sequences encoding hASIC1B can be identified by the absence of marker gene function. Alternatively, a marker gene can be placed in tandem with a sequence encoding hASIC1B under the control of a single promoter. Expression of the marker gene in response to induction or selection usually indicates expression of the tandem gene as well.

Alternatively, host cells, which contain the coding sequence for hASIC1B and express hASIC1B may be identified by a variety of procedures known to those of skill in the art. These procedures include, but are not limited to, DNA-DNA or DNA-RNA hybridizations and protein bioassay or immunoassay techniques, which include membrane, solution, or chip based technologies for the detection and/or quantification of the nucleic acid or protein.

The presence of the polynucleotide sequence encoding hASIC1B can be detected by DNA-DNA or DNA-RNA hybridization or amplification using probes or portions or fragments of polynucleotides encoding hASIC1B. Nucleic acid amplification based assays involve the use of oligonucleotides or oligomers based on the hASIC1B-encoding sequence to detect transformants containing DNA or RNA encoding hASIC1B. As used herein “oligonucleotides” or “oligomers” refer to a nucleic acid sequence of at least about 10 nucleotides and as many as about 60 nucleotides, preferably about 15 to 30 nucleotides, and more preferably about 20-25 nucleotides, which can be used as a probe or amplimer.

A variety of protocols for detecting and measuring the expression of hASIC1B, using either polyclonal or monoclonal antibodies specific for the protein are known in the art. Examples include enzyme-linked immunosorbent assay (ELISA), radioimmunoassay (RIA), and fluorescent activated cell sorting (FACS). A two-site, monoclonal-based immunoassay utilizing monoclonal antibodies reactive to two non-interfering epitopes on hASIC1B is preferred, but a competitive binding assay may be employed. These and other assays are described, among other places, in “Serological Methods: A Laboratory Manual”, Hampton et al., APS Press, 1990, St-Paul, Mich. and Maddox et al., J Exp Med 1983; 158: 1211).

A wide variety of labels and conjugation techniques are known by those skilled in the art and may be used in various nucleic acid and amino acid assays. Means for producing labeled hybridization or PCR probes for detecting sequences related to polynucleotides encoding hASIC1B include oligolabeling, nick translation, end-labeling or PCR amplification using a labeled nucleotide. Alternatively, the sequence encoding hASIC1B, or any portion of it, may be cloned into a vector for the production of an mRNA probe. Such vectors are known in the art, are commercially available, and may be used to synthesize RNA probes in vitro by addition of an appropriate RNA polymerase such as T7, T3 or SP6 and labeled nucleotides. These procedures may be conducted using a variety of commercially available kits: from e.g. Pharmacia & Upjohn, (Kalamazoo, Mich.); Promega (Madison Wis.); and U.S. Biochemical Corp. (Cleveland, Ohio), or Ambion (Austin, Tex.). Suitable reporter molecules or labels, which may be used, include radionuclides, enzymes, fluorescent, chemiluminescent, or chromogenic agents as well as substrates, cofactors, inhibitors, magnetic particles, and the like.

Host cells transformed with a nucleotide sequence encoding hASIC1B may be cultured under conditions suitable for the expression and recovery of the protein from cell culture. The protein produced by a recombinant cell may be secreted or contained intracellularly depending on the sequence and/or the vector used. As will be understood by those of skill in the art, expression vectors containing polynucleotides, which encode hASIC1B may be designed to contain signal sequences which direct secretion of hASIC1B through a prokaryotic or eukaryotic cell membrane. Other recombinant constructions may be used to join sequences encoding hASIC1B to nucleotide sequence encoding a polypeptide domain, which will facilitate purification of soluble proteins. Such purification facilitating domains include, but are not limited to, metal chelating peptides such as histidine-tryptophan modules that allow purification on immobilized metals, protein A domains that allow purification on immobilized immunoglobulin, and the domain utilized in the FLAGS extension/affinity purification system (Immunex Corp., Seattle, Wash.). The inclusion of cleavable linker sequences such as those specific for Factor XA or enterokinase (Invitrogen, San Diego, Calif.) between the purification domain and hASIC1B may be used to facilitate purification. One such expression vector provides for expression of a fusion protein containing hASIC1B, a thioredoxin or an enterokinase cleavage site, and followed by six histidine residues. The histidine residues facilitate purification on IMIAC (immobilized metal ion affinity chromatography as described in Porath et al., Prot Exp Purif 1992; 3: 263) while the enterokinase cleavage site provides a means for purifying hASIC1B from the fusion protein. A discussion of vectors which contain fusion proteins is provided in Kroll et al. (DNA Cell Biol 1993; 12: 441).

In addition to recombinant production, fragments of hASIC1B may be produced by direct peptide synthesis using solid-phase techniques (see Stewart et al., “Solid-Phase Peptide Synthesis”, WH Freeman & Co., 1969, San Francisco, Calif.; Merrifield et al., J Am Chem Soc 1963; 85: 2149). Chemical synthesis may be performed using manual techniques or by automation. Automated synthesis may be achieved, for example, using Applied Biosystems 431A Peptide Synthesizer (Perkin Elmer). Various fragments of hASIC1B may be chemically synthesized separately and combined using chemical methods to produce the full-length molecule.

Thus, as set forth herein, the invention includes the provision of a novel subfamily of proton-gated channel proteins as exemplified by the novel DNA sequences set for the in FIG. 1 (SEQ ID NO: 1), as well as DNA sequences which hybridize thereto under hybridization conditions of the stringency equal to or greater than the conditions of the stringency employed in the initial isolation of cDNAs of the invention, and DNA sequences encoding the same allelic variant or analog proton-gated channel protein through use of at least in part degenerate codons. The sequences can also be used to located and identify other closely related members of this subfamily as described in Cannessa et al (Nature 1994; 367: 463).

The novel protein products of the invention include polypeptides with the primary structural conformation (i.e. amino acid sequence) of proton-gated channel proteins as set froth in FIG. 1 and SEQ ID NO:2, as well as peptide fragments thereof and synthetic peptides assembled to be duplicative of amino acid sequences thereof. Proteins, protein fragments and synthetic proteins or peptides of the invention are projected to have uses earlier described including therapeutic, diagnostic, and prognostic assays and protocols and will provide the basis for monoclonal and polyclonal antibodies specifically reactive with the channel protein.

Therapeutics

In another embodiment of the invention, hASIC1B or fragments thereof may be used for therapeutic purposes. Based on the chemical and structural homology among hASIC1B (SEQ ID NO: 2) and other ASIC receptors (FIG. 2), and RT-PCR and Northern blot analysis showing that hASIC1B transcripts are primarily but not exclusively associated with cells of the peripheral and central nervous systems, hASIC1B is believed to play a role in the regulation of neurotransmitter release, neuronal excitability, excitotoxicity or mecanosensation. Indeed, secretory granules and synaptic vesicules are known to contain high concentrations of protons (low intravesicular pH), which are co-released with other neurotransmifters during regulated and constitutive exocytosis. Released protons might thus activate pre- and/or post-synaptic, or extrasynaptic hASIC1B receptors. Indeed, under certain conditions, low pH or extracellular acidosis has been shown to influence synaptic transmission as well as the induction of long-term potentiation (Igelmund et al., Brain Res 1995; 689: 9; Velisek et al., Hippocampus 1998; 8: 24). Also, in certain animal seizure models, neuroprotective effects of low pH have been observed (Velisek et al., Exp Brain Res 1994; 101: 44). ASIC 2A has been directly implicated in mecanodetection using knockout animals. Furthermore, the particular repeating structures in the first exon of hASC1B may be indicative of some specific function, such as interaction with or anchoring to the cytoskeleton. hASIC1B might therefore constitute a crucial component of a mecanogated ion channel. Thus, an important use of hASIC1B is screening for compounds that regulate neurotransmitter release, synaptic efficacy, neuroexcitability, or neurotoxicity. Such compounds may have utility in a number of physiological and pathological situations pertaining, for example, to cognition, perception, learning, memory, pain and many others.

In one embodiment, antagonists or inhibitors of the protein or vectors expressing antisense sequences may be used to treat disorders and diseases of the nervous system resulting from altered ion transport, signal transmission, and apoptosis. Such diseases include, but are not limited to, chronic pain, inflammatory pain, neuropathic pain such as diabetic-, cancer-, and AIDS-related, neurodegenerative diseases such as Alzheimer's disease, Parkinson's disease, Huntington's disease, Creutzfeld-Jacob disease, and amyotrophic lateral sclerosis, and dementias, including AIDS-related, as well as convulsions, epilepsy, stroke, and anxiety and depression.

In another embodiment, antagonists or inhibitors of the protein or vectors expressing antisense sequences may be used to treat cardiovascular diseases such as angina, congestive heart failure, vasoconstriction, hypertension, atherosclerosis, restenosis, and bleeding.

In another embodiment, antagonists or inhibitors of the protein or vectors expressing antisense sequences may be used to treat disorders and diseases of the reproductive system, in particular male infertility, or may also be used as male contraceptive agents.

Agonists which enhance the activity and antagonists which block or modulate the effect of hASIC1B may be used in those situations where such enhancement or inhibition is therapeutically desirable. Such agonists, antagonists or inhibitors may be produced using methods which are generally known in the art, and particularly involve the use of purified hASIC1B to produce antibodies or to screen libraries of pharmaceutical agents for those which specifically bind hASIC1B. For example, in one aspect, antibodies which are specific for hASIC1B may be used directly as an antagonist, or indirectly as a targeting or delivery mechanism for bringing a pharmaceutical agent to cells or tissue which express hASIC1B.

The antibodies may be generated using methods that are well known in the art. Such antibodies may include, but are not limited to, polyclonal, monoclonal, chimeric, single chain, Fab fragments, and fragments produced by a Fab expression library. Neutralizing antibodies, (i.e., those which inhibit dimer formation) are especially preferred for therapeutic use.

For the production of antibodies, various hosts including goats, rabbits, rats, mice, humans, and others, may be immunized by injection with hASIC1B or any fragment or oligopeptide thereof which has immunogenic properties. Depending on the host species, various adjuvants may be used to increase immunological response. Such adjuvants include, but are not limited to, Freund's, mineral gels such as aluminum hydroxide, and surface active substances such as lysolecithin, pluronic polyols, polyanions, peptides, oil emulsions, keyhole limpet hemocyanin, and dinitrophenol. Among adjuvants used in humans, BCG (bacilli Calmette-Guerin) and Corynebacterium parvum are especially preferable.

It is preferred that the peptides, fragments, or oligopeptides used to induce antibodies to hASIC1B have an amino acid sequence consisting of at least five amino acids, and more preferably at least 10 amino acids. It is also preferable that they are identical to a portion of the amino acid sequence of the natural protein, and they may contain the entire amino acid sequence of a small, naturally occurring molecule. Short stretches of hASIC1B amino acids may be fused with those of another protein such as keyhole limpet hemocyanin and antibody produced against the chimeric molecule.

Monoclonal antibodies to hASIC1B may be prepared using any technique which provides for the production of antibody molecules by continuous cell lines in culture. These include, but are not limited to, the hybridoma technique, the human B-cell hybridoma technique, and the EBV-hybridoma technique (Koehler et al. Nature 1975; 256: 495; Kosbor et al., Immunol Today 1983; 4: 72; Cote et al., Proc Natl Acad Sci 1983; 80: 2026; Cole et al., “Monoclonal Antibodies and Cancer Therapy”, Alan R. Liss Inc., 1985, New York, N.Y., pp. 77-96).

In addition, techniques developed for the production of “chimeric antibodies”, the splicing of mouse antibody genes to human antibody genes to obtain a molecule with appropriate antigen specificity and biological activity can be used (Morrison et al. (1984) Proc. Natl. Acad. Sci. 81:6851-6855; Neuberger et al. (1984) Nature 312:604-608; Takeda et al. (1985) Nature 314:452-454). Alternatively, techniques described for the production of single chain antibodies may be adapted, using methods known in the art, to produce hASIC1B-specific single chain antibodies. Antibodies with related specificity, but of distinct idiotypic composition, may be generated by chain shuffling from random combinatorial immunoglobin libraries (Burton, D. R. (1991) Proc. Natl. Acad. Sci. 88:11120-3).

Antibodies may also be produced by inducing in vivo production in the lymphocyte population or by screening recombinant immunoglobulin libraries or panels of highly specific binding reagents as disclosed in the literature (Orlandi et al. (1989) Proc. Natl. Acad. Sci. 86: 3833-3837; Winter, G. et al. (1991) Nature 349:293-299).

Antibody fragments which contain specific binding sites for hASIC1B may also be generated. For example, such fragments include, but are not limited to, the F(ab′)₂ fragments which can be produced by pepsin digestion of the antibody molecule and the Fab fragments which can be generated by reducing the disulfide bridges of the F(ab′)₂ fragments. Alternatively, Fab expression libraries may be constructed to allow rapid and easy identification of monoclonal Fab fragments with the desired specificity (Huse et al. (1989) Science 256:1275-1281).

Various immunoassays may be used for screening to identify antibodies having the desired specificity. Numerous protocols for competitive binding or immunoradiometric assays using either polyclonal or monoclonal antibodies with established specificities are well known in the art. Such immunoassays typically involve the measurement of complex formation between hASIC1B and its specific antibody. A two-site, monoclonal-based immunoassay utilizing monoclonal antibodies reactive to two non-interfering hASIC1B epitopes is preferred, but a competitive binding assay may also be employed (Maddox, supra).

In another embodiment of the invention, the polynucleotides encoding hASIC1B, or any fragment thereof, or antisense sequences, may be used for therapeutic purposes. In one aspect, antisense to the polynucleotide encoding hASIC1B may be used in situations in which it would be desirable to block the synthesis of the protein. In particular, cells may be transformed with sequences complementary to polynucleotides encoding hASIC1B. Thus, antisense sequences may be used to modulate hASIC1B activity, or to achieve regulation of gene function. Such technology is now well known in the art, and sense or antisense oligomers or larger fragments, can be designed from various locations along the coding or control regions of sequences encoding hASIC1B.

Expression vectors derived from retroviruses, adenovirus, herpes or vaccinia viruses, or from various bacterial plasmids may be used for delivery of nucleotide sequences to the targeted organ, tissue or cell population. Methods, which are well known to those skilled in the art, can be used to construct recombinant vectors which will express antisense polynucleotides of the gene encoding hASIC1B. These techniques are described both in Sambrook et al. (supra) and in Ausubel et al. (supra).

Genes encoding hASIC1B can be turned off by transforming a cell or tissue with expression vectors that express high levels of a polynucleotide or fragment thereof which encodes hASIC1B. Such constructs may be used to introduce untranslatable sense or antisense sequences into a cell. Even in the absence of integration into the DNA, such vectors may continue to transcribe RNA molecules until all copies are disabled by endogenous nucleases.

Transient expression may last for a month or more with a non-replicating vector and even longer if appropriate replication elements are part of the vector system.

As mentioned above, modifications of gene expression can be obtained by designing antisense molecules, DNA, RNA or PNA, to the control regions of the gene encoding hASIC1B, i.e., the promoters, enhancers, and introns. Oligonucleotides derived from the transcription initiation site, e.g., between positions −10 and +10 from the 5′ end of the transcript, are preferred. Similarly, inhibition can be achieved using “triple helix” base-pairing methodology. Triple helix pairing is useful because it causes inhibition of the ability of the double helix to open sufficiently for the binding of polymerases, transcription factors, or regulatory molecules. Recent therapeutic advances using triplex DNA have been described in the literature (Gee, J. E. et al. (1994) In: Huber, B. E. and Carr, B. I. Molecular and Immunologic Approaches, Futura Publishing Co., Mt. Kisco, N.Y.). The antisense molecules may also be designed to block translation of mRNA by preventing the transcript from binding to ribosomes.

Ribozymes, enzymatic RNA molecules, may also be used to catalyze the specific cleavage of RNA. The mechanism of ribozyme action involves sequence-specific hybridization of the ribozyme molecule to complementary target RNA, followed by endonucleolytic cleavage. Examples which may be used include engineered hammerhead motif ribozyme molecules that can specifically and efficiently catalyze endonucleolytic cleavage of sequences encoding hASIC1B.

Specific ribozyme cleavage sites within any potential RNA target are initially identified by scanning the target molecule for ribozyme cleavage sites which include the following sequences: GUA, GUU, and GUC. Once identified, short RNA sequences of between 15 and 20 ribonucleotides corresponding to the region of the target gene containing the cleavage site may be evaluated for secondary structural features which may render the oligonucleotide inoperable. The suitability of candidate targets may also be evaluated by testing accessibility to hybridization with complementary oligonucleotides using ribonuclease protection assays.

Antisense molecules and ribozymes of the invention may be prepared by any method known in the art for the synthesis of RNA molecules. These include techniques for chemically synthesizing oligonucleotides such as solid phase phosphoramidite chemical synthesis. Alternatively, RNA molecules may be generated by in vitro and in vivo transcription of DNA sequences encoding hASIC1B. Such DNA sequences may be incorporated into a wide variety of vectors with suitable RNA polymerase promoters such as T7 or SP6. Alternatively, these cDNA constructs that synthesize antisense RNA constitutively or inducibly can be introduced into cell lines, cells, or tissues.

RNA molecules may be modified to increase intracellular stability and half-life. Possible modifications include, but are not limited to, the addition of flanking sequences at the 5′ and/or 3′ ends of the molecule or the use of phosphorothioate or 2′ O-methyl rather than phosphodiesterase linkages within the backbone of the molecule. This concept is inherent in the production of PNAs and can be extended in all of these molecules by the inclusion of nontraditional bases such as inosine, queosine, and wybutosine, as well as acetyl-, methyl-, thio-, and similarly modified forms of adenine, cytidine, guanine, thymine, and uridine which are not as easily recognized by endogenous endonucleases.

Many methods for introducing vectors into cells or tissues are available and equally suitable for use in vivo, in vitro, and ex vivo. For ex vivo therapy, vectors may be introduced into stem cells taken from the patient and clonally propagated for autologous transplant back into that same patient. Delivery by transfection and by liposome injections may be achieved using methods that are well known in the art.

Any of the therapeutic methods described above may be applied to any suitable subject including, for example, mammals such as dogs, cats, cows, horses, rabbits, monkeys, and most preferably, humans.

An additional embodiment of the invention relates to the administration of a pharmaceutical composition, in conjunction with a pharmaceutically acceptable carrier, for any of the therapeutic effects discussed above. Such pharmaceutical compositions may consist of hASIC1B, antibodies to hASIC1B, mimetics, agonists, antagonists, or inhibitors of hASIC1B. The compositions may be administered alone or in combination with at least one other agent, such as stabilizing compound, which may be administered in any sterile, biocompatible pharmaceutical carrier, including, but not limited to, saline, buffered saline, dextrose, and water. The compositions may be administered to a patient alone, or in combination with other agents, drugs or hormones.

The pharmaceutical compositions utilized in this invention may be administered by any number of routes including, but not limited to, oral, intravenous, intramuscular, intra-arterial, intramedullary, intrathecal, intraventricular, transdermal, subcutaneous, intraperitoneal, intranasal, enteral, topical, sublingual, or rectal means. In addition to the active ingredients, these pharmaceutical compositions may contain suitable pharmaceutically-acceptable carriers comprising excipients and auxiliaries which facilitate processing of the active compounds into preparations which can be used pharmaceutically. Further details on techniques for formulation and administration may be found in the latest edition of Remington's Pharmaceutical Sciences (Maack Publishing Co., Easton, Pa.).

Pharmaceutical compositions for oral administration can be formulated using pharmaceutically acceptable carriers well known in the art in dosages suitable for oral administration. Such carriers enable the pharmaceutical compositions to be formulated as tablets, pills, dragees, capsules, liquids, gels, syrups, slurries, suspensions, and the like, for ingestion by the patient.

Pharmaceutical preparations for oral use can be obtained through combination of active compounds with solid excipient, optionally grinding a resulting mixture, and processing the mixture of granules, after adding suitable auxiliaries, if desired, to obtain tablets or dragee cores. Suitable excipients are carbohydrate or protein fillers, such as sugars, including lactose, sucrose, mannitol, or sorbitol; starch from corn, wheat, rice, potato, or other plants; cellulose, such as methyl cellulose, hydroxypropylmethyl-cellulose, or sodium carboxymethylcellulose; gums including arabic and tragacanth; and proteins such as gelatin and collagen. If desired, disintegrating or solubilizing agents may be added, such as the cross-linked polyvinyl pyrrolidone, agar, alginic acid, or a salt thereof, such as sodium alginate.

Dragee cores may be used in conjunction with suitable coatings, such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound, i.e., dosage.

Pharmaceutical preparations that can be used orally include push-fit capsules made of gelatin, as well as soft, sealed capsules made of gelatin and a coating, such as glycerol or sorbitol. Push-fit capsules can contain active ingredients mixed with a filler or binders, such as lactose or starches, lubricants, such as talc or magnesium stearate, and, optionally, stabilizers. In soft capsules, the active compounds may be dissolved or suspended in suitable liquids, such as fatty oils; liquid, or liquid polyethylene glycol with or without stabilizers.

Pharmaceutical formulations suitable for parenteral administration may be formulated in aqueous solutions, preferably in physiologically compatible buffers such as Hanks's solution, Ringer's solution, or physiologically buffered saline. Aqueous injection suspensions may contain substances, which increase the viscosity of the suspension, such as sodium carboxymethyl cellulose, sorbitol, or dextran. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Optionally, the suspension may also contain suitable stabilizers or agents which increase the solubility of the compounds to allow for the preparation of highly concentrated solutions.

For topical or nasal administration, penetrants appropriate to the particular barrier to be permeated are used in the formulation. Such penetrants are generally known in the art.

The pharmaceutical compositions of the present invention may be manufactured in a manner that is known in the art, e.g., by means of conventional mixing, dissolving, granulating, dragee-making, levigating, emulsifying, encapsulating, entrapping, or lyophilizing processes.

The pharmaceutical composition may be provided as a salt and can be formed with many acids, including but not limited to, hydrochloric, sulfuric, acetic, lactic, tartaric, malic, succinic, etc. Salts tend to be more soluble in aqueous or other protonic solvents than are the corresponding free base forms. In other cases, the preferred preparation may be a lyophilized powder which may contain any or all of the following: 1-50 mM histidine, 0.1%-2% sucrose, and 2-7% mannitol, at a pH range of 4.5 to 5.5, that is combined with buffer prior to use.

After pharmaceutical compositions have been prepared, they can be placed in an appropriate container and labeled for treatment of an indicated condition. For administration of hASIC1B, such labeling would include amount, frequency, and method of administration. Pharmaceutical compositions suitable for use in the invention include compositions wherein the active ingredients are contained in an effective amount to achieve the intended purpose. The determination of an effective dose is well within the capability of those skilled in the art.

For any compound, the therapeutically effective dose can be estimated initially either in cell culture assays, e.g., of neoplastic cells, or in animal models, usually mice, rabbits, dogs, or pigs. The animal model may also be used to determine the appropriate concentration range and route of administration. Such information can then be used to determine useful doses and routes for administration in humans. A therapeutically effective dose refers to that amount of active ingredient, for example hASIC1B or fragments thereof, antibodies of hASIC1B, agonists, antagonists or inhibitors of hASIC1B, which ameliorates the symptoms or condition. Therapeutic efficacy and toxicity may be determined by standard pharmaceutical procedures in cell cultures or experimental animals, e.g., ED50 (the dose therapeutically effective in 50% of the population) and LD50 (the dose lethal to 50% of the population). The dose ratio between therapeutic and toxic effects is the therapeutic index, and it can be expressed as the ratio, LD50/ED50.

Pharmaceutical compositions, which exhibit large therapeutic indices are preferred. The data obtained from cell culture assays and animal studies is used in formulating a range of dosage for human use. The dosage contained in such compositions is preferably within a range of circulating concentrations that include the ED50 with little or no toxicity. The dosage varies within this range depending upon the dosage form employed, sensitivity of the patient, and the route of administration.

The exact dosage will be determined by the practitioner, in light of factors related to the subject that requires treatment. Dosage and administration are adjusted to provide sufficient levels of the active moiety or to maintain the desired effect. Factors, which may be taken into account include the severity of the disease state, general health of the subject, age, weight, and gender of the subject, diet, time and frequency of administration, drug combination(s), reaction sensitivities, and tolerance/response to therapy. Long-acting pharmaceutical compositions may be administered every 3 to 4 days, every week, or once every two weeks depending on half-life and clearance rate of the particular formulation.

Normal dosage amounts may vary from 0.1 to 100,000 micrograms, up to a total dose of about 1 g, depending upon the route of administration. Guidance as to particular dosages and methods of delivery is provided in the literature and generally available to practitioners in the art. Those skilled in the art will employ different formulations for nucleotides than for proteins or their inhibitors. Similarly, delivery of polynucleotides or polypeptides will be specific to particular cells, conditions, locations, etc.

Diagnostics

In another embodiment, antibodies that specifically bind hASIC1B may be used for the diagnosis of conditions or diseases characterized by expression of hASIC1B, or in assays to monitor patients being treated with hASIC1B, agonists, antagonists or inhibitors. The antibodies useful for diagnostic purposes may be prepared in the same manner as those described above for therapeutics. Diagnostic assays for hASIC1B include methods that utilize the antibody and a label to detect hASIC1B in human body fluids or extracts of cells or tissues. The antibodies may be used with or without modification, and may be labeled by joining them, either covalently or non-covalently, with a reporter molecule. A wide variety of reporter molecules, which are known in the art may be used, several of which are described above.

A variety of protocols including ELISA, RIA, and FACS for measuring hASIC1B are known in the art and provide a basis for diagnosing altered or abnormal levels of hASIC1B expression. Normal or standard values for hASIC1B expression are established by combining body fluids or cell extracts taken from normal mammalian subjects, preferably human, with antibody to hASIC1B under conditions suitable for complex formation The amount of standard complex formation may be quantified by various methods, but preferably by photometric, means. Quantities of hASIC1B expressed in subject, control and disease, samples from biopsied tissues are compared with the standard values. Deviation between standard and subject values establishes the parameters for diagnosing disease.

In another embodiment of the invention, the polynucleotides encoding hASIC1B may be used for diagnostic purposes. The polynucleotides, which may be used include oligonucleotide sequences, antisense RNA and DNA molecules, and PNAs. The polynucleotides may be used to detect and quantitate gene expression in biopsied tissues in which expression of hASIC1B may be correlated with disease. The diagnostic assay may be used to distinguish between absence, presence, and excess expression of hASIC1B, and to monitor regulation of hASIC1B levels during therapeutic intervention.

In one aspect, hybridization with PCR probes which are capable of detecting polynucleotide sequences, including genomic sequences, encoding hASIC1B or closely related molecules, may be used to identify nucleic acid sequences which encode hASIC1B. The specificity of the probe, whether it is made from a highly specific region, e.g., 10 unique nucleotides in the 5′ regulatory region, or a less specific region, e.g., especially in the 3′ coding region, and the stringency of the hybridization or amplification (maximal, high, intermediate, or low) will determine whether the probe identifies only naturally occurring sequences encoding hASIC1B, alleles, or related sequences.

Probes may also be used for the detection of related sequences, and should preferably contain at least 50% of the nucleotides from any of the hASIC1B encoding sequences. The hybridization probes of the subject invention may be DNA or RNA and derived from the nucleotide sequence of SEQ ID NO: 1 or from genomic sequence including promoter, enhancer elements, and introns of the naturally occurring hASIC1B.

Means for producing specific hybridization probes for DNAs encoding hASIC1B include the cloning of nucleic acid sequences encoding hASIC1B or hASIC1B derivatives into vectors for the production of mRNA probes. Such vectors are known in the art, commercially available, and may be used to synthesize RNA probes in vitro by means of the addition of the appropriate RNA polymerases and the appropriate labeled nucleotides. Hybridization probes may be labeled by a variety of reporter groups, for example, radionuclides such as 32P or 35S, or enzymatic labels, such as alkaline phosphatase coupled to the probe via avidin/biotin coupling systems, and the like. Polynucleotide sequences encoding hASIC1B may be used for the diagnosis of conditions or diseases that are associated with expression of hASIC1B. Examples of such conditions or diseases include neurological diseases including chronic pain, neuropathic pain such as diabetic-, cancer-, and AIDS-related, neurodegenerative diseases such as Alzheimer's disease, Parkinson's disease, Huntington's disease, Creutzfeld-Jacob disease, and amyotrophic lateral sclerosis, and dementias, such as AIDS-related, as well as convulsions, epilepsy, stroke, and anxiety and depression, cardiovascular diseases such as angina, congestive heart failure, vasoconstriction, hypertension, atherosclerosis, restenosis, and bleeding. The polynucleotide sequences encoding hASIC1B may be used in Southern or northern analysis, dot blot, or other membrane-based technologies; in PCR technologies; or in dip stick, pin, ELISA or chip assays utilizing fluids or tissues from patient biopsies to detect altered hASIC1B expression. Such qualitative or quantitative methods are well known in the art

In a particular aspect, the nucleotide sequences encoding hASIC1B may be useful in assays that detect activation or induction of various neurological or other non-neurological disorders, particularly those mentioned above. The nucleotide sequence encoding hASIC1B may be labeled by standard methods, and added to a fluid or tissue sample from a patient under conditions suitable for the formation of hybridization complexes. After a suitable incubation period, the sample is washed and the signal is quantitated and compared with a standard value. If the amount of signal in the biopsied or extracted sample is significantly altered from that of a comparable control sample, the nucleotide sequence has hybridized with nucleotide sequences in the sample, and the presence of altered levels of nucleotide sequences encoding hASIC1B in the sample indicates the presence of the associated disease. Such assays may also be used to evaluate the efficacy of a particular therapeutic treatment regimen in animal studies, in clinical trials, or in monitoring the treatment of an individual patient.

In order to provide a basis for the diagnosis of disease associated with expression of hASIC1B, a normal or standard profile for expression is established. This may be accomplished by combining body fluids or cell extracts taken from normal subjects, either animal or human, with a sequence, or a fragment thereof, which encodes hASIC1B, under conditions suitable for hybridization or amplification. Standard hybridization may be quantified by comparing the values obtained from normal subjects with those from an experiment where a known amount of a substantially purified polynucleotide is used. Standard values obtained from normal samples may be compared with values obtained from samples from patients who are symptomatic for disease. Deviation between standard and subject values is used to establish the presence of disease.

Once disease is established and a treatment protocol is initiated, hybridization assays may be repeated on a regular basis to evaluate whether the level of expression in the patient begins to approximate that which is observed in the normal patient. The results obtained from successive assays may be used to show the efficacy of treatment over a period ranging from several days to months.

With respect to neurological diseases, the presence of a relatively high amount of transcript in biopsied tissue from an individual may indicate a predisposition for the development of the disease, or may provide a means for detecting the disease prior to the appearance of actual clinical symptoms. A more definitive diagnosis of this type may allow health professionals to employ preventative measures or aggressive treatment earlier thereby preventing the development or further progression of the disease.

Additional diagnostic uses for oligonucleotides encoding hASIC1B may involve the use of PCR. Such oligomers may be chemically synthesized, generated enzymatically, or produced from a recombinant source. Oligomers will preferably consist of two nucleotide sequences, one with sense orientation and another with antisense, employed under optimized conditions for identification of a specific gene or condition. The same two oligomers, nested sets of oligomers, or even a degenerate pool of oligomers may be employed under less stringent conditions for detection and/or quantitation of closely related DNA or RNA sequences.

Methods which may also be used to quantitate the expression of hASIC1B include radiolabeling or biotinylating nucleotides, coamplification of a control nucleic acid, and standard curves onto which the experimental results are interpolated (Melby, P. C. et al. (1993) J. Immunol. Methods, 159:235-244; Duplaa, C. et al. (1993) Anal. Biochem. 229-236). The speed of quantitation of multiple samples may be accelerated by running the assay in an ELISA format where the oligomer of interest is presented in various dilutions and a spectrophotometric or colorimetric response gives rapid quantitation.

In another embodiment of the invention, the nucleic acid sequence that encodes hASIC1B may also be used to generate hybridization probes that are useful for mapping the naturally occurring genomic sequence. The sequence may be mapped to a particular chromosome or to a specific region of the chromosome using well known techniques. Such techniques include FISH, FACS, or artificial chromosome constructions, such as yeast artificial chromosomes, bacterial artificial chromosomes, bacterial P1 constructions or single chromosome cDNA libraries as reviewed by Price, C. M. (1993; Blood Rev. 7:127-134), and Trask, B. J. (1991; Trends Genet. 7:149-154).

FISH (as described in Verma et al. (1988) Human Chromosomes: A Manual of Basic Techniques, Pergamon Press, New York, N.Y.) may be correlated with other physical chromosome mapping techniques and genetic map data. Examples of genetic map data can be found in the 1994 Genome Issue of Science (265:1981f). Correlation between the location of the gene encoding hASIC1B on a physical chromosomal map and a specific disease, or predisposition to a specific disease, may help delimit the region of DNA associated with that genetic disease. The nucleotide sequences of the subject invention may be used to detect differences in gene sequences between normal, carrier, or affected individuals.

In situ hybridization of chromosomal preparations and physical mapping techniques such as linkage analysis using established chromosomal markers may be used for extending genetic maps. Often the placement of a gene on the chromosome of another mammalian species, such as mouse, may reveal associated markers even if the number or arm of a particular human chromosome is not known. New sequences can be assigned to chromosomal arms, or parts thereof, by physical mapping. This provides valuable information to investigators searching for disease genes using positional cloning or other gene discovery techniques. Once the disease or syndrome has been crudely localized by genetic linkage to a particular genomic region, for example, AT to 11q22-23 (Gatti et al. (1988) Nature 336:577-580), any sequences mapping to that area may represent associated or regulatory genes for further investigation. The nucleotide sequence of the subject invention may also be used to detect differences in the chromosomal location due to translocation, inversion, etc. among normal, carrier, or affected individuals.

Screening Assays

In another embodiment of the invention, hASIC1B, its catalytic or immunogenic fragments or oligopeptides thereof, can be used for screening libraries of compounds in any of a variety of drug screening techniques. The fragment employed in such screening may be free in solution, affixed to a solid support, borne on a cell surface, or located intracellularly. The formation of binding complexes, between hASIC1B and the agent being tested, may be measured. Thus, the ppolypeptides of the invention may also be used to assess the binding of small molecule substrates and ligands in, for example, cells, cell-free preparations, chemical libraries, and natural product mixtures. These substrates and ligands may be natural substrates and ligands or may be structural or functional mimetics. In general, such screening procedures involve producing appropriate cells, which express the receptor ploypeptide of the present invention on the surface thereof. Such cells include cells from mammals, yeast, insects (eg Drosophila) or bacteria (eg E. coli). Cells expressing the receptor (or cell membranes containing the expressed receptor) are then contacted with a test compound to observe binding, or stimulation or inhibition of a functional respone (for example inhibition of proton-activated currents).

The assays my simply test binding of a candidate compound wherein adherence to the cells bearing the receptor is detected by means of a label directly or indirectly associated with the candidate compound or in an assay involving competition with a labeled competitor. Further, these assays may test whether the candidate compound results in a signal generated by activation of the receptor, using detection systems appropriate to the cells bearing the receptor at their surfaces (for example increased ion permeation measured by patch clamp or, preferably by ion imaging). Inhibitors of activation are generally assayed in the presence of a known agonist (for example, protons) and the effect of the candidate compound on the activation by the agonist is observed. Standard methods for conducting such screening assays are well understood in the art. Typically, the response may be measured by use of a microelectrode technique accompanied by such measurement strategies as voltage clamping of the cell whereby activation of ion channels may be identified by inward or outward current flow as detected using the microelectrodes. ²²Na, ⁸⁶Rb, ⁴⁵Ca radiolabeled cations or ¹⁴C or ³H guanidine may be used to assess such ion flux; a sodium, calcium or potassium ion sensitive dye (such as Fura-2, or Indo) may also be used to monitor ion passage through the receptor ion channel, or a potential sensitive dye may be used to monitor potential changes, such as in depolarization.

Alternatively, it is also possible to mutate the hASIC1B cDNA in order to produce a constituvely active hASIC1B channel, as has been shown with other DEG/EnaC family members (Huang et al., Nature 367: 467; Waldman et al., J Biol Chem 1997: 271; 10433). Then, the constitutively active channel may be expressed in host cells to produce a screening assay where channel activity is permanent. The recording of channel activity my be carried out either by membrane voltage analysis, directly (patch clamp, for example) or indirectly (fluorescent probes, for example) or by sodium entry measurement (radioactive sodium influx, fluorescent probes, or reporter genes).

Another technique for drug screening, which may be used provides for high throughput screening of compounds having suitable binding affinity to the protein of interest as described in published PCT application WO84/03564. In this method, as applied to hASIC1B large numbers of different small test compounds are synthesized on a solid substrate, such as plastic pins or some other surface. The test compounds are reacted with hASIC1B, or fragments thereof, and washed. Bound hASIC1B is then detected by methods well known in the art. Purified hASIC1B can also be coated directly onto plates for use in the aforementioned drug screening techniques. Alternatively, non-neutralizing antibodies can be used to capture the peptide and immobilize it on a solid support.

In another embodiment, one may use competitive drug screening assays in which neutralizing antibodies capable of binding hASIC1B specifically compete with a test compound for binding hASIC1B. In this manner, the antibodies can be used to detect the presence of any peptide which shares one or more antigenic determinants with hASIC1B.

In additional embodiments, the nucleotide sequences that encode hASIC1B may be used in any molecular biology techniques that have yet to be developed, provided the new techniques rely on properties of nucleotide sequences that are currently known, including, but not limited to, such properties as the triplet genetic code and specific base pair interactions.

### EXAMPLES

The following examples are intended to further illustrate the invention and are not intended to limit the scope of the invention in any way. All references cited herein, whether previously or in the following examples, are expressly incorporated in their entirety by reference.

### Example 1

### Cloning of Full Length cDNA Encoding the hASIC1B Protein

A blast search of the entire GenBank database using selected polypeptide motifs caracteristic of rat ASIC1B N-terminal polypeptide, retrieved a human genomic polynucleotide molecule (GenBank accession: AC025154, AC074032, AC025361). Subsequent methodological analysis of the above cited genomic DNA sequence based on sequence comparison and alignment with cloned ASIC family members as well as consensus intron/exon splicing sites allowed the identification of a cDNA sequence encoding a novel human ASIC subunit, herein named hASIC1B. The alignment with the published rat ASIC1b immediately revealed that both receptors differed at the 5-prime end and that the initiating methionine on the human receptor was not evident. The identified human sequence contained three potential initiation sites (see FIG. 1 ) and accordingly three putative hASIC1B constructs were prepared. Functional analysis revealed that only the longest version of the hASIC1B receptor was functional and therefore constitutes the actual human receptor, as confirmed by RT-PCR. Alignment of the coding regions of ASIC receptors reveal that hASIC1B initiation segment shows similarities to the hASIC4 region. The actual construction of the hASIC1B was achieved by RT-PCR amplification of the 5-prime end of hASIC1B from reverse transcribed human trigeminal ganglion cDNA using specific oligonucleotide primers of SEQ ID NO: 3 and SEQ ID NO: 4. The Polymerase Chain Reaction (PCR) was performed with the EXPAND long-template polymerase mix, containing both Taq and Pwo polymerases (Roche Diagnostics, formerly from Boehringer Mannheim). Reaction conditions followed the manufacturers instructions. Briefly, reaction mix included: dNTPs 0.5 mM, forward and reverse primers 1 μM each, RT-cDNA template 5 μL, 10×PCR buffer 5 μL and polymerase enzyme mix 0.75 μL, all in a final volume of 50 μL. Samples were kept at 4° C. and the enzyme mix was added last. Tubes were then immediately transferred to the thermocycler preheated to 94° C., after which cycling was launched. Typical cycling conditions were as follows: Initial denaturation step: 2 min at 94° C., than 40 cycles of 45 sec at 94° C., 45 sec at 58° C. and 2 min at 72° C., followed by a final extension step of 10 min at 72° C. RT-cDNAs from human trigenimal ganglia was prepared from RNA or mRNA with the Superscript or Thermoscript enzyme mix according to the manufacturers directions (Gibco Life Sciences). RNA and mRNA were prepared using standard molecular biology protocols, such as decribed in Maniatis et al., (see above) or using commecially available kits, such as for example the S.N.A.P. total RNA isolation kit, Fast Track 2.0 and micro Fast Track 2.0 mRNA isolation kits (InVitrogen). The amplified fragment was subsequently purified and restriction digested with HindIII and NotI and then ligated to the 3-prime fragment of hASIC1A which is the common region to both splice variants.

### Example 2

### Labeling and Use of Hybridization Probes

Hybridization probes derived from SEQ ID NO: 1 are employed to screen cDNAs, genomic DNAs, or mRNAs. Although the labeling of oligonucleotides, consisting of about 20 base-pairs, is specifically described, essentially the same procedure is used with larger cDNA fragments. Oligonucleotides are designed using state-of-the-art software such as GeneWorks 2.5.1 (Oxford Molecular), labeled by combining 50 pmol of each oligomer and 250 μCi of γ³²P adenosine triphosphate (Amersham) and T4 polynucleotide kinase (DuPont NEN, Boston, Mass.). The labeled oligonucleotides are substantially purified with Sephadex G-25 superfine resin column (Pharmacia & Upjohn). Labelled sense and antisense oligonucleotides are then used in a typical membrane based hybridization analysis of human genomic DNA digested with one of the following endonucleases (Ase I, Bgl II, Eco RI, Pst I, Xba I or Pvu II; DuPont NEN).

The DNA from each digest is fractionated on a 0.7% agarose gel and transferred to nylon membranes (Nytran Plus, Schleicher & Schuell, Durham, N. H.). Hybridization is carried out for 16 hours at 40° C. To remove nonspecific signals, blots are sequentially washed at room temperature under increasingly stringent conditions up to 0.1× saline sodium citrate and 0.5% sodium dodecyl sulfate. After XOMAT AR film (Kodak, Rochester, N.Y.) is exposed to the blots in a Phosphoimager cassette (Molecular Dynamics, Sunnyvale, Calif.) for several hours, hybridization patterns are compared visually.

### Example 3

### Northern Blot Analysis

Northern analysis is a laboratory technique used to detect the presence of a transcript of a gene and involves the hybridization of a labeled nucleotide sequence to a membrane on which RNAs from a particular cell type or tissue have been bound (Sambrook et al., supra).

Northern blots containing 2 μg of poly(A)+RNA isolated from specific adult human tissues or from sections of the brain are obtained from commercial sources (Clontech). Probes are prepared by random prime labeling (Pharmacia Biotech Inc.), or as described above. PCR primers (SEQ ID NO: 5 and SEQ ID NO: 6) specific for the 5′ and 3′ ends of the protein coding sequence of the first exon of hASIC1B cDNA were used in a PCR reaction to generate a fragment containing the entire hhASIC1B-specific sequence. This fragment was cloned into the pBluescript vector and used to probe the multiple tissue blots. Filters were hybridized overnight at 42° C. in a buffer containing 50% formamide, 5×SSPE, 2% SDS, 10× Denhardt's solution, and 100 μg/ml salmon sperm DNA. Filters were washed with 0.1×SSC, 0.1% SDS at 55° C. and exposed to Kodak X-Omat AR film for 4 days at −70° C. Results indicate that hASIC1B is not detectable in peripheral tissues and is mainly expressed in neuronal tissues. In the brain, hASIC1B is expressed mainly in the thalamus, caudal nucleus, the p.g. of the cortex, cerebellum, substantia nigra, medula oblongata, putamen and lower levels in the corpus collosum.

### Example 4

### Expression of hASIC1B in Xenopus laevis Oocytes

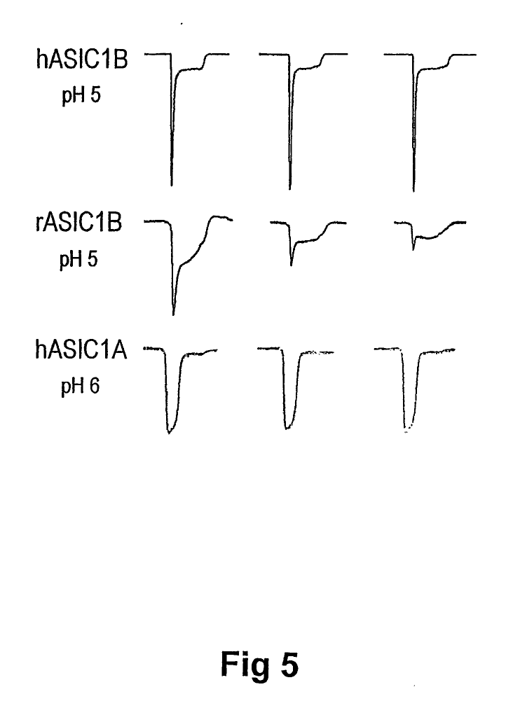

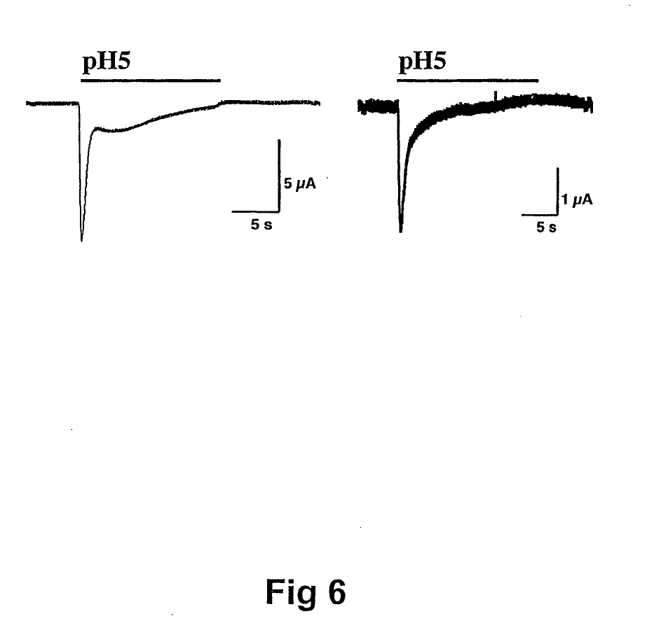

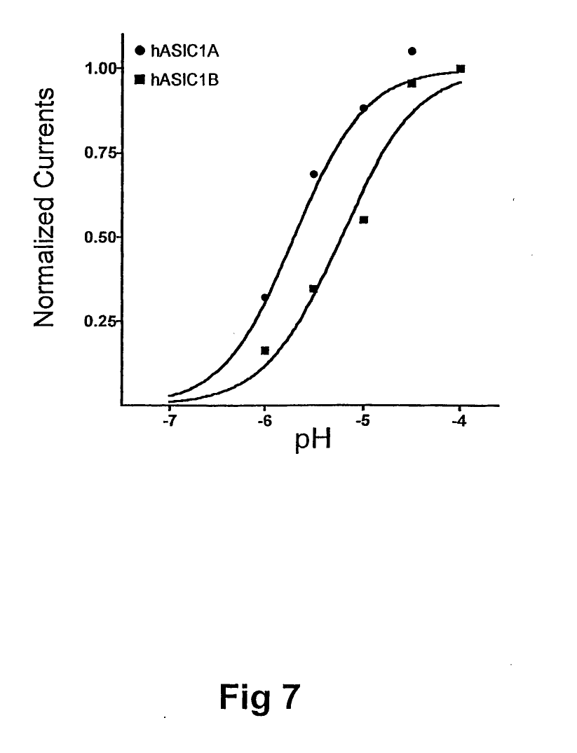

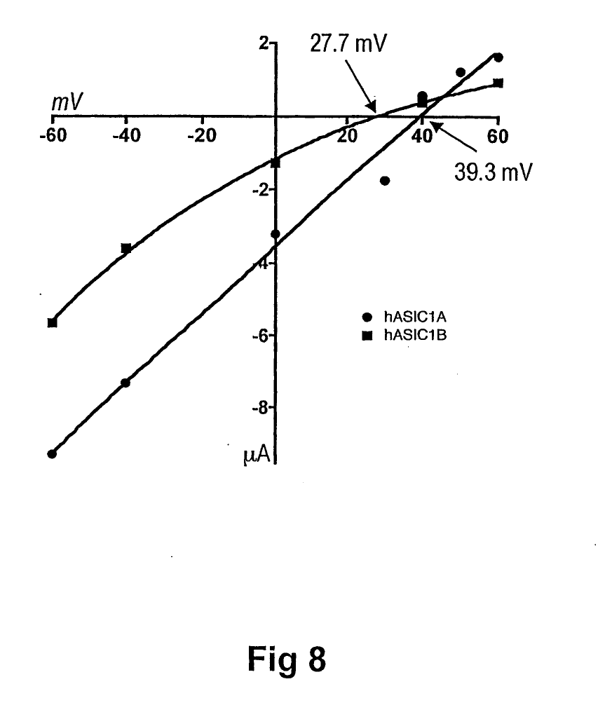

hASIC1B is expressed in Xenopus oocytes by nuclear injection of hASIC1B cDNA subcloned into pCDNA3 (1-5 ng). Control oocytes were injected with H₂O. Oocytes were maintained at 18° C. in modified Barth's solution. Current was measured by two-electrode voltage clamp 1-3 days after injection. During voltage clamp (−60 mV/−100 mV)), oocytes were bathed in 116 mM NaCl, 2 mM KCl, 1.8 mM CaCl₂, 10 mM acetic acid and 5 mM Hepes (pH 7.4 with NaOH). To determine proton-gating, bath solution was quickly switched to a solution of pH 5 for 10 sec, then returned to bath solution for washout. The stimulating solution was prepared by lowering the pH of the original bath solution with hydrochloric acid. The osmolality of the solutions was verified with an osmometer and corrected with mannitol or choline chloride. To document ionic selectivity, NaCl was replaced with LiCl or KCl. Current-voltage relationships were determined by stepping from a holding potential of −60 mV to potentials between −100 and +60 mV for 10 seconds before and during simulation with low pH solution. Similar functional charaterizations are also performed in patch-clamp with COS cells transfected with the hASIC1B containing pcDNA vector. Typical current in response to pH 5 is shown in FIG. 5 and FIG. 6, respectively with COS cells and oocytes. Dose-response curves with varying pH (FIG. 7) as well as I/V curves (FIG. 8) for hASIC1B channels were obtained in a similar fashion.

### Example 5

### Non-Functional Dominant-Negative hASIC1B Subunit Generated by Site-Directed Muragenesis

DEG/EnaC ion channel subunits associate into homo and/or heteromultumeric complexes, which form the actual channel. It is therefore possible by artificially modifying specific amino acids in a given sequence to render a particular subunit non-functional regarding, for example, channel activity, but still retaining its ability to interact with other subunits. The resulting complex, which comprises such non-functional mutant, also becomes inactive, even in the presence of agonist. Amino acids, which are targetted, are preferably highly conserved throughout a given family of ion channel subunits. We have targeted certain amino acids of the ASIC family and found that a conserved Gly residue (i.e position 439 in human ASIC3, or position 469 in hASIC1B), when substituted with an Arg residue, generates a dominant-negative mutant. When the hASIC3-G439R is expressed alone in oocytes, no current is observed in response to low pH under voltage clamp conditions. Furthermore, when the mutant is coinjected with a wild type ASIC3 subunit, no functional channel is expressed because of the dominant-negative effect of the G439R mutant. The substitution of Gly469 from hASIC1B to an Arg residue is done using the commercially available mutagenesis kit “QuickChange” (Stragene), according to the kit's directives. Briefly, two antiparallel oligonucleotide primers, each complementary to opposite strands in the same region, are synthesized carrying the desired mutation as a single mismatch: a “C” replaces the “G” in the first position of the codon encoding Gly469, the rest of the nucleotides being identical. The oligonucleotide primers are then extended during temperature cycling by PfuTurbo DNA polymerase using as template a pCDNA3 plasmid comprising the cDNA encoding the full length hASIC1B. On incorporation of the oligonucleotide primers, a mutated plasmid containing staggered nicks is generated. After temperature cycling, the product is treated with Dpn I. The Dpn I is used to digest the parental DNA template and select for the synthesized DNA containing mutations. Since DNA isolated from most E. coli strains is dam methylated, it is susceptible to Dpn I digestion, which is specific for methylated and hemimethylated DNA. The nicked vector DNA incorporating the desired mutations is then transformed into E. coli.

Once the correct mutation has been confirmed by sequencing, the mutant hASIC1B is tested in oocytes or mammamlian expression systems, as described herein.

Such dominant-negative mutants may be used as tools to investigate the different combinations of subunit interactions, and to study physiological role of hASIC1B and its involvement pathophysiological conditions by breeding transgenic animals carrying the dominant-negative mutant. These mutants can also be used as an alternative to antisense oligonucleotides, for example in gene therapy.

### Example 6

### Extension of hASIC1B-Encoding Polynucleotides to Full Length or to Recover Regulatory Sequences

Full length hASIC1B-encoding nucleic acid sequence (SEQ ID NO: 1) is used to design oligonucleotide primers for extending a partial nucleotide sequence to full length or for obtaining 5′ or 3′, intron or other control sequences from genomic libraries. One primer is synthesized to initiate extension in the antisense direction (R) and the other is synthesized to extend sequence in the sense direction (F). Primers are used to facilitate the extension of the known sequence “outward” generating amplicons containing new, unknown nucleotide sequence for the region of interest. The initial primers are designed from the cDNA using GeneWorks 5.0.1 (Oxford Molecular, Cambridge, UK), or another appropriate program, to be 22-30 nucleotides in length, to have a GC content of 50% or more, and to anneal to the target sequence at temperatures about 68.degree.-72.degree. C. Any stretch of nucleotides which would result in hairpin structures and primer-primer dimerizations is avoided.

The original, selected cDNA libraries, or a human genomic library are used to extend the sequence; the latter is most useful to obtain 5′ upstream regions. If more extension is necessary or desired, additional sets of primers are designed to further extend the known region.

By following the instructions for the EXPAND long template PCR kit (Roche Diagnostics) and thoroughly mixing the enzyme and reaction mix, high fidelity amplification is obtained. Beginning with 40 pmol of each primer and the recommended concentrations of all other components of the kit, PCR is performed using the Peltier Thermal Cycler (PTC200; M.J. Research, Watertown, Mass.) and the following parameters:

| | |
|---|---|
| Step 1 | 94° C. for 1 min (initial denaturation) |
| Step 2 | 65° C. for 1 min |
| Step 3 | 68° C. for 6 min |
| Step 4 | 94° C. for 15 sec |
| Step 5 | 65° C. for 1 min |
| Step 6 | 68° C. for 7 min |
| Step 7 | Repeat step 4-6 for 15 additional cycles |
| Step 8 | 94° C. for 15 sec |
| Step 9 | 65° C. for 1 min |
| Step 10 | 68° C. for 7:15 min |
| Step 11 | Repeat step 8-10 for 12 cycles |
| Step 12 | 72° C. for 8 min |
| Step 13 | 4° C. (and holding) |

A 5-10 uL aliquot of the reaction mixture is analyzed by electrophoresis on a low concentration (about 0.6-0.8%) agarose mini-gel to determine which reactions were successful in extending the sequence. Bands thought to contain the largest products are selected and removed from the gel. Further purification involves using a commercial gel extraction method such as QIAQuick.TM. (QIAGEN Inc., Chatsworth, Calif.). After recovery of the DNA, Klenow enzyme is used to trim single-stranded, nucleotide overhangs creating blunt ends which facilitate religation and cloning.

After ethanol precipitation, the products are redissolved in 13 uL of ligation buffer, 1 μl T4-DNA ligase (15 units) and 1 μl T4 polynucleotide kinase are added, and the mixture is incubated at room temperature for 2-3 hours or overnight at 16.degree. C. Competent E. coli cells (in 40 μl of appropriate media) are transformed with 3 μl of ligation mixture and cultured in 80 μl of SOC medium (Sambrook et al., supra). After incubation for one hour at 37° C., the whole transformation mixture is plated on Luria Bertani (LB)-agar (Sambrook et al., supra) containing 2.times. Carb. The following day, several colonies are randomly picked from each plate and cultured in 150 μl of liquid LB/2.times. Carb medium placed in an individual well of an appropriate, commercially-available, sterile 96-well microtiter plate. The following day, 5 μl of each overnight culture is transferred into a non-sterile 96-well plate and after dilution 1:10 with water, 5 μl of each sample is transferred into a PCR array.

For PCR amplification, 18 μl of concentrated PCR reaction mix (3.3.times.) containing 4 units of rTth DNA polymerase, a vector primer, and one or both of the gene specific primers used for the extension reaction are added to each well. Amplification is performed using the following conditions:

| | |
|---|---|
| Step 1 | 94° C. for 60 sec |
| Step 2 | 94° C. for 20 sec |
| Step 3 | 55° C. for 30 sec |
| Step 4 | 72° C. for 90 sec |
| Step 5 | Repeat steps 2-4 for an additional 29 cycles |
| Step 6 | 72° C. for 180 sec |
| Step 7 | 4° C. (and holding) |

Aliquots of the PCR reactions are run on agarose gels together with molecular weight markers. The sizes of the PCR products are compared to the original partial cDNAs, and appropriate clones are selected, ligated into plasmid, and sequenced.

### Example 7

### Antisense Molecules

Antisense molecules to the hASIC1B-encoding sequence, or any part thereof, is used to inhibit in vivo or in vitro expression of naturally occurring hASIC1B. Although use of antisense oligonucleotides, comprising about 20 base-pairs, is specifically described, essentially the same procedure is used with larger cDNA fragments. An oligonucleotide based on the coding sequences of hASIC1B, as shown in FIG. 1, is used to inhibit expression of naturally occurring hASIC1B. The complementary oligonucleotide is designed from the most unique 5′ sequence as shown in FIG. 1 and used either to inhibit transcription by preventing binding to the upstream untranscribed sequence or translation of an hASIC1B-encoding transcript by preventing ribosomes from binding. Using an appropriate portion of the 5′ sequence of SEQ ID NO: 1, an effective antisense oligonucleotide includes any 15-20 nucleotides spanning the region which translates into the 5′ coding sequence of the polypeptide as shown in FIG. 1.

### Example 8

### Expression of hASIC1B

Expression of hASIC1B is accomplished by subcloning the cDNA into appropriate vectors and transforming the vectors into host cells. In this case, the cloning vector, pSport is used to express hASIC1B in E. coli. Upstream of the cloning site, this vector contains a promoter for β-galactosidase, followed by sequence containing the amino-terminal Met, and the subsequent seven β-galactosidase. Immediately following these eight residues is a bacteriophage promoter useful for transcription and a linker containing a number of unique restriction sites.

Induction of an isolated, transformed bacterial strain with IPTG using standard methods produces a fusion protein which consists of the first eight residues of β-galactosidase, about 5 to 15 residues of linker, and the full length protein. The signal residues direct the secretion of hASIC1B into the bacterial growth Media which can be used directly in the following assay for activity.

### Example 9

### Production of hASIC1B Specific Antibodies

hASIC1B that is substantially purified using PAGE electrophoresis (Sambrook, supra), or other purification techniques, is used to immunize rabbits and to produce antibodies using standard protocols. The amino acid sequences deduced from SEQ ID NO: 2 are analyzed using MacVector 6.0.1 (oxford Molecular) to determine regions of high immunogenicity and a corresponding oligopolypeptide is synthesized and used to raise antibodies by means known to those of skill in the art. Selection of appropriate epitopes, such as those near the C-terminus or in hydrophilic regions, is described by Ausubel et al. (supra), and others.

Typically, the oligopeptides are 15 residues in length, synthesized using an Applied Biosystems Peptide Synthesizer Model 431 A using fmoc-chemistry, and coupled to keyhole limpet hemocyanin (KLH, Sigma, St. Louis, Mo.) by reaction with N-maleimidobenzoyl-N-hydroxysuccinimide ester (MBS; see “Antobodies: A Laboratory Manual”, Harlow E and Lane D, eds., 1998, CSHL Press, Plainview, N.Y). Rabbits are immunized with the oligopeptide-KLH complex in complete Freund's adjuvant. The resulting antisera are tested for antipeptide activity, for example, by binding the peptide to plastic, blocking with 1% BSA, reacting with rabbit antisera, washing, and reacting with radioiodinated, goat anti-rabbit IgG.

### Example 10

### Purification of Naturally Occurring hASIC1B Using Specific Antibodies

Naturally occurring or recombinant hASIC1B is substantially purified by immunoaffinity chromatography using antibodies specific for hASIC1B. An immunoaffinity column is constructed by covalently coupling hASIC1B antibody to an activated chromatographic resin, such as CnBr-activated Sepharose (Pharmacia & Upjohn). After the coupling, the resin is blocked and washed according to the manufacturers instructions.

Media containing hASIC1B is passed over the immunoaffinity column, and the column is washed under conditions that allow the preferential absorbance of hASIC1B (e.g., high ionic strength buffers in the presence of detergent). The column is eluted under conditions that disrupt antibody/hASIC1B binding (e.g., a buffer of pH 2-3 or a high concentration of a chaotrope, such as urea or thiocyanate ion), and hASIC1B is collected.

### Example 11

### Identification of Molecules Which Interact with hASIC1B

Permanently or transiently transfected COS cell lines in multiwell plates expressing hASIC1B are loaded with potential-sensitive dyes and the fluorescence emission is measured following application of a low pH buffer (pH 5.0) The responses in the presence and absence of candidate compounds is compared to identify compounds which stimulate, inhibit or modulate hASIC1B.

Alternatively, hASIC1B or biologically active fragments thereof are labeled with ¹²⁵I-Bolton-Hunter reagent (Bolton et al. (1973) Biochem. J. 133:529). Candidate molecules previously arrayed in the wells of a multi-well plate are incubated with the labeled hASIC1B, washed and any wells with labeled hASIC1B complex are assayed. Data obtained using different concentrations of hASIC1B are used to calculate values for the number, affinity, and association of hASIC1B with the candidate molecules.

All publications mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments.

1. A purified and isolated human proton-gated ion channel protein (hASIC1B) selected from the following: a hASIC1B having at least 80% identity with the amino acid sequence defined in SEQ ID NO:2, a hASIC1B having at least 85% identity with the amino acid sequence defined in SEQ ID NO:2, a hASIC1B having at least 90% identity with the amino acid sequence defined in SEQ ID NO:2, a hASIC1B having at least 95% identity with the amino acid sequence defined in SEQ ID NO:2, a hASIC1B having at least 98% identity with the amino acid sequence defined in SEQ ID NO:2 and a hASIC1B having at least 99% identity with the amino acid sequence defined in SEQ ID NO:2.

2. The protein of claim 1, which is characterized by the activation of protons (acids, low pH solutions).

3. The protein of claim 2 which has the amino acid sequence defined in SEQ ID NO:2.

4. A nucleic acid which encodes a protein as defined in claim 1.

5. The nucleic acid of claim 4, which is capable of hybridizing to SEQ ID NO: 1.

6. The nucleic acid of claim 4, which has the sequence defined in SEQ ID NO: 1.

7. A recombinant vector, comprising the nucleic acid of claim 4.

8. The recombinant vector of claim 7, which is an expression vector.

9. A host comprising the recombinant vector of claim 7.

10. A host cell comprising the recombinant vector of claim 8.

11. A process for producing a human proton-gated ion channel protein (hASIC1B) comprising culturing the host cell of claim 10 under conditions sufficient for the production of said protein and recovering said protein from the culture.

12. A process for producing a cell which produces a protein as defined in claim 1.

13. A process as defined in claim 12, further comprising transforming or transfecting a host cell with a recombinant expression vector comprising a nucleic acid which encodes a protein, wherein the protein (hASIC1B) is selected from the following: a hASIC1B having at least 80% identity with the amino acid sequence defined in SEQ ID NO:2, a hASIC1B having at least 85% identity with the amino acid sequence defined in SEQ ID NO:2, a hASIC1B having at least 90% identity with the amino acid sequence defined in SEQ ID NO:2, a hASIC1B having at least 95% identity with the amino acid sequence defined in SEQ ID NO:2, a hASIC1B having at least 98% identity with the amino acid sequence defined in SEQ ID NO:2 and a hASIC1B having at least 99% identity with the amino acid sequence defined in SEQ ID NO:2.

14. An antibody immunospecific for a hASIC1B protein as defined in claim 1.

15. A hybridoma producing an antibody as defined in claim 14.

16. A method for the treatment of a subject in need of enhanced activity or expression of a hASIC1B protein, wherein the protein is selected from the following: a hASIC1B having at least 80% identity with the amino acid sequence defined in SEQ ID NO:2, a hASIC1B having at least 85% identity with the amino acid sequence defined in SEQ ID NO:2, a hASIC1B having at least 90% identity with the amino acid sequence defined in SEQ ID NO:2, a hASIC1B having at least 95% identity with the amino acid sequence defined in SEQ ID NO:2, a hASIC1B having at least 98% identity with the amino acid sequence defined in SEQ ID NO:2 and a hASIC1B having at least 99% identity with the amino acid sequence defined in SEQ ID NO:2, comprising
administering to the subject a therapeutically effective amount of an agonist to said hASIC1B protein; or
providing to the subject a nucleic acid of claim 4 in a form so as to effect production of said hASIC1B protein activity in vivo.

17. A method for the treatment of a subject having a need to inhibit activity or expression of a hASIC1B protein of claim 1 comprising:
(a) administering to the subject a therapeutically effective amount of an antagonist to said hASIC1B protein; or
(b) administering to the subject a nucleic acid molecule that inhibits the expression of the nucleotide sequence encoding said hASIC1B protein; or
(c) administering to the subject a therapeutically effective amount of a protein that competes with said hASIC1B protein for its ligand.

18. A process for diagnosing a disease or a susceptibility to a disease in a subject related to the expression or activity of a hASIC1B protein as defined in any of claim 1 in a subject comprising:
(a) determining the presence or absence of a mutation in the nucleotide sequence encoding said hASIC1B protein in the genome of said subject; or
(b) analyzing for the presence or amount of hASIC1B protein expression in a sample derived from said subject.

19. A method for identifying agonists to a hASIC1B protein, wherein the hASIC1B protein is selected from the following: a hASIC1B having at least 80% identity with the amino acid sequence defined in SEQ ID NO:2, a hASIC1B having at least 85% identity with the amino acid sequence defined in SEQ ID NO:2, a hASIC1B having at least 90% identity with the amino acid sequence defined in SEQ ID NO:2, a hASIC1B having at least 95% identity with the amino acid sequence defined in SEQ ID NO:2, a hASIC1B having at least 98% identity with the amino acid sequence defined in SEQ ID NO:2 and a hASIC1B having at least 99% identity with the amino acid sequence defined in SEQ ID NO:2, comprising:
(a) putting cells produced by the process of claim 11 in contact with candidate compound(s); and
(b) determining whether the candidate compound induces or modulates a biological activity or signal transduced by the hASIC1B receptor; or
(c) determining whether the candidate compound induces inward currents or modulates proton-induced inward currents transduced by the hASIC1B receptor.

20. An agonist identified by the method of claim 19.

21. An agonist of claim 20, which is, or is an adjuvant to, an antidepressant, a desensitizing agent, an antipruritic, an analgesic, a chemotherapeutic or antineoplastic agent, an antipsychotic, a psychotherapeutic agent, a respiratory and cerebral stimulant, a cognitive stimulant, memory stimulant, a promoter of neuronal regeneration, a stimulant of cell growth or proliferation, an insecticide, a pesticide or an anthelmintic, or any combination thereof.

22. The method for identifying antagonists to a hASIC1B protein, wherein the hASIC1B protein is selected from the following: a hASIC1B having at least 80% identity with the amino acid sequence defined in SEQ ID NO:2, a hASIC1B having at least 85% identity with the amino acid sequence defined in SEQ ID NO:2, a hASIC1B having at least 90% identity with the amino acid sequence defined in SEQ ID NO:2, a hASIC1B having at least 95% identity with the amino acid sequence defined in SEQ ID NO:2, a hASIC1B having at least 98% identity with the amino acid sequence defined in SEQ ID NO:2 and a hASIC1B having at least 99% identity with the amino acid sequence defined in SEQ ID NO:2, comprising:
(a) putting cells produced by the process of claim 11 in contact with a low pH solution (pH<7.4) or any other agonist; and
(b) determining whether the signal generated by protons or said agonist is modulated, diminished or abolished in the presence of candidate compound(s).

23. An antagonist identified by the method of claim 22.

24. An antagonist as defined in claim 23, which is, or is an adjuvant to, an analgesic, an antipyretic, an antipruritic, an anxiolytic, sedative or hypnotic, a psychotherapeutic agent, an anticonvulsant, a neuroprotectant, a general anesthetic, a local anesthetic, a hypotensive agent, a muscle relaxant, an antidiarrhea agent, an antacid, or any combination thereof.


SEQUENCE LISTING (2 sequences)

SEQ ID NO: 1 - DNA, 3658 bp, human
  CDS (1)..(1686)
atgcccatccagatcttctgctccatgtcattctcctctggagaggaagccccagggcccttgggagatatttggggtccccaccaccatcagcagcagcaggacatctcagaatcggaagaggaggaagaagagaaggaaaaggaggcagtgaggaaggaggccagtgaggggcattcacccatggacttggtggcctttgccaacagctgcaccctccatggcaccaaccacatttttgtggaggggggtccagggccaaggcaggtgctgtggtcggtggcctttgtcctggcactgggtgccttcctgtgccaggtaggggaccgcgttgcttattacctcagctacccacacgtgacccttctaaacgaagtggccaccacggagctggccttcccagcagtcaccctctgcaacactaatgctgtgcggctgtcccagctcagctaccctgacttgctttatttggcccccatgctgggactggatgaaagtgatgaccccggggtgcccctcgctccaccgggccctgaggccttctctggggagccctttaacctgcaccgcttctacaatcgctcctgccaccggctggaggacatgctgctctattgctcctaccaagggggaccctgcggccctcacaacttctcagtggtcttcacacgctatggaaagtgctacacgttcaactcgggccgagatgggcggccgcggctgaagaccatgaaggatgggacgggcaatgggctggaaatcatgctggacatccagcaggacgagtacctgcctgtgtggggggagactgacgagacgtccttcgaagcaggcatcaaagtgcagatccatagtcaggatgaacctcctttcatcgaccagctgggctttggcgtggccccaggcttccagacctttgtggcctgccaggagcagcggctcatctacctgcccccaccctggggcacctgcaaagctgttaccatggactcggatttggatttcttcgactcctacagcatcactgcctgccgcatcgactgtgagacgcgctacctggtggagaactgcaactgccgcatggtgcacatgccaggggatgccccatactgtactccagagcagtacaaggagtgtgcagatcctgctctggacttcctggtggagaaggaccaggagtactgcgtgtgtgaaatgccttgcaacctgacccgctatggcaaagagctgtccatggtcaagatccccagcaaagcctcagccaagtacctggccaagaagttcaacaaatctgagcaatacataggggagaacatcctggtgctggacattttctttgaagtcctcaactatgagaccattgaacagaagaaggcctatgagattgcagggctcctgggtgacatcgggggccagatggggctgttcatcggggccagcatcctcacggtgctggagctctttgactacgcctacgaggtcattaagcacaagctgtgccgacgaggaaaatgccagaaggaggccaaaaggagcagtgcggacaagggcgtggccctcagcctggacgacgtcaaaagacacaacccgtgcgagagccttcggggccaccctgccgggatgacatacgctgccaacatcctacctcaccatccggcccgaggcacgttcgaggactttacctgctgagccccgcaggccgctgaaccaaaggcctagatggggaggactaggagagcgrgggggcccccagctgcctcctcacatctgccctgggractccccacactccggggcagatctttcctcttgtctgtggtaaggaaggagtcttgaccatagagtcctctctctgcctctatcccattcyttttacatttaacaaaactaatctaaaaaagaactaaaaagggagaacggggcaagggacctcaggctgcccctctctcctccatgctgcctcccctagctcccagcctgaattctgtctatctagctgtctgccatctgagtgtccatctacattctgctgccaccagtcaccaaaggcccttcccagtgaggggtggaagggatctctggggtctggaatttggccccaaaccagagaatgtaccttaagggggagggctagtgtgggggagggaggcttccccagccttaagagaccctctcagcccagtgactgtccccaaacccaagtctcctggcaggaactaaaacctcagccccactctctcacaccatgtggaatctcgtgggggtcggggatccccttaagaagtggtaatggggacaagatgcggccctggtgctgtaggctacatcctgatacctataagttcacccccaccccacagctgctggagagaaatcccaagaggcagcccttcctcaccatcccattaaagacckggctggttagcgtccagctcagggagaagggcgctagtgcctaacctcactggtccctctcccggaggcccttgtagagggccacgtccataaattttcttatggaactctcccacatcctcttccccaacttcatttgcttctctcaacaacctcatctgcattttctatttctatatgatacagactctatattgctatatctctgtatatactttcccagccctgtctgtctccaccccatcccctcttgtctctgagaaccattctcccaccccaagttccaccttctatgtttctactccctccctggtctctgaatgccttygcctgtataaagagttggactctctcccctggtgtctgtactgtgtacacacatccctctgagaagcacaaggagacgacacgcgcattgtaacctttgcactgtctcagtggcgacaaaggaagctgtgaatcacaagctctgcctctttctggcctcaccctctcccccaacccgggcaccctcggccctccctgcagccttaacattctcttcccctgctcctcctatcccattgccctctgcccagctgacagtggcatccccagggaaggggttgctgtagagatagcccccacccaggggatggaggtctaccctggacactaagccaagtgtgtcagagacagaagggagctggggattggcgactcctgaagttggggcagtgggatgctgacaggcagaagctgaggtcctcagtcagtggcctttcctccttctgggtgcccagccccctttcctcacctgatacccaagcccaccacttttattttctggtgaggtgggtttgggaggaaagagaggcctagaggaggagttgaaagctctgctgttgtctcaccctatcttaatgagagacaagtgaggtggagggcctgccccccctccctccaccagacactccttccaggcctgagccccaacccctcttcaggccttccttccctagctgtgtcttggtcttcaatcccagaacaggacctgtgagcagctgcattggcctggagctggagagtaaggctgtaggatctttggaatctcttggttcctaagagtttcctcagagatcatacctccccagagggaagcaggaatgaggccaaaaagtgtgcattggataggggaacagcaggcagggctctgggtgacgcatgcctctggtctaataaactgggtttcaaccaaaaaaaa

SEQ ID NO: 2 - protein, 562 aa, human
MPIQIFCSMSFSSGEEAPGPLGDIWGPHHHQQQQDISESEEEEEEKEKEAVRKEASEGHSPMDLVAFANSCTLHGTNHIFVEGGPGPRQVLWSVAFVLALGAFLCQVGDRVAYYLSYPHVTLLNEVATTELAFPAVTLCNTNAVRLSQLSYPDLLYLAPMLGLDESDDPGVPLAPPGPEAFSGEPFNLHRFYNRSCHRLEDMLLYCSYQGGPCGPHNFSVVFTRYGKCYTFNSGRDGRPRLKTMKDGTGNGLEIMLDIQQDEYLPVWGETDETSFEAGIKVQIHSQDEPPFIDQLGFGVAPGFQTFVACQEQRLIYLPPPWGTCKAVTMDSDLDFFDSYSITACRIDCETRYLVENCNCRMVHMPGDAPYCTPEQYKECADPALDFLVEKDQEYCVCEMPCNLTRYGKELSMVKIPSKASAKYLAKKFNKSEQYIGENILVLDIFFEVLNYETIEQKKAYEIAGLLGDIGGQMGLFIGASILTVLELFDYAYEVIKHKLCRRGKCQKEAKRSSADKGVALSLDDVKRHNPCESLRGHPAGMTYAANILPHHPARGTFEDFTC